(19) United States
(12) Patent Application Publication
(10) Pub. No.: US 2010/0319595 A1
(43) Pub. Date: Dec. 23, 2010

(54) Method for the production of thermally advanced feedlot biomass (TAFB) for use as fuel

(75) Inventors: John M. Sweeten, Amarillo, TX (US); Kalyan Annamalai, College Station, TX (US); Kevin Heflin, Amarillo, TX (US); Brent W. Auvermann, Amarillo, TX (US); Lanny A. McDonald, Amarillo, TX (US); L. Wayne Greene, Auburn, AL (US); Doug Tolleson, Cottonwood, AZ (US)

Correspondence Address: CONLEY ROSE, P.C.;David A. Rose, P. O. BOX 3267, HOUSTON, TX, 77253-3267, US

(73) Assignee: The Texas A&M University System, College Station, TX (US)

(21) Appl. No.: 12/152,374

(22) Filed: May 14, 2008

Related U.S. Application Data
(60) Provisional application No. 60/917,820, filed on May 14, 2007.

(51) Int. Cl. F23G 5/02; F23G 7/00; C10L 5/42; C12P 1/04

(52) U.S. Cl. 110/342; 44/605; 435/170; 44/500; 44/504

(57) ABSTRACT

A method for the production of animal feedlot biomass for use as fuel in a reactor comprises surfacing a feedlot with a feedlot surfacing material. In addition, the method comprises collecting an animal feedlot biomass from the feedlot. Further, the method comprises preparing the collected biomass for use as fuel.

### CROSS-REFERENCE TO RELATED APPLICATIONS

This application claims benefit of U.S. provisional application Ser. No. 60/917,820 filed May 14, 2007, and entitled “Method for the Production of Thermally Advanced Feedlot Biomass (TAFB) for Use as Fuel,” which is hereby incorporated herein by reference in its entirety.

### STATEMENT REGARDING FEDERALLY SPONSORED RESEARCH OR DEVELOPMENT

The subject technology was developed in part under research contracts from the U.S. Department of Energy, Contract Number DE-FG26-00NT40810 and Contract Number DE-FG36-05G085003; and USDA-CSREES/North Carolina State University Grant Number 93-36200-8701.

### BACKGROUND

1. Field of the Invention

The disclosure relates generally to the production of thermally advanced feedlot biomass for use as fuel. More specifically, the disclosure relates to the production of enhanced feedlot biomass from feedlots (e.g. outdoor feedlots), the biomass having improved properties for combustion, gasification, and/or bioconversion.

### BACKGROUND OF THE INVENTION

Animal waste disposal is a major problem being faced by nations around the globe. Cattle feedlot manure is primarily used as fertilizer with a very small amount currently used for energy production. New policy and economic incentives, as well as improved technologies, may lead to greater use of feedlot manure as a renewable energy feedstock. The use of cattle manure as organic soil fertilizer for farmland applications may result in higher amounts of phosphorus in the soil than can be utilized by the crops. The runoff of this excess phosphorus accumulated in the soil may result in water eutrophication. Due to the severity of this and other problems associated with animal waste disposal, the industry is seeking environmentally friendly methods to dispose of animal wastes. Feedlot biomass (FB) has been identified as a low-pollution, zero net carbon dioxide fuel, which may be used to produce energy in an environmentally-friendly manner.

Approximately 2% of cattle on feed are fed under roof on concrete floors in confinement buildings from which manure is collected either in solid or semi-solid form with or without supplemental absorptive bedding at intervals of several days, weeks, or months. Alternatively, depending on pen floor design, the manure may be collected daily or more often in a liquid (slurry) form using water as a carrier or as a semisolid using mechanical scrapers. Unless excessive water content can be economically removed, the resulting semisolid or liquid manure generally is more suitable for biological conversion (e.g., anaerobic digestion, etc.) than for thermal conversion.

More than 98% of the 11 million head of feedlot cattle currently in the U.S. are fed outdoors in conventional open-soil surfaced feedpens in which the manure is subjected to weathering and normally is collected (harvested) from the pen surface following each turn of cattle after a feeding period of approximately 130-160 days per head. On an as-collected basis, feedlot cattle generate manure at the rate of approximately one ton per head on feed for slaughter. Consequently, the amount of manure collected is approximately 2 tons per head of feedlot capacity per year. This amount depends upon, among other factors, ration digestibility and the extent of soil entrained with the manure.

Conventionally collected feedlot biomass in solid form has been shown to have potential physical characteristics that may be suitable for conversion to energy forms, including primary combustion via co-firing, secondary combustion (e.g., reburn), gasification, or other means of thermal conversion. However, the characteristics of feedlot manure, insofar as its utility as a fuel, vary widely depending on the conditions of collection, storage, and subsequent handling. Feedlot biomass having a high amount of ash may result in ash fouling when used in existing pulverized fuel-fired boilers. In addition to utility as fuel, feedlot biomass also has utility for bioconversion, such as fermentation and anaerobic digestion to produce biogas significantly comprising methane, carbon dioxide, or combinations thereof.

Accordingly, a need in the art exists exists for methods of producing thermally advanced feedlot biomass that is enhanced for use as fuel for combustion including co-firing and reburn, gasification, pyrolysis, and other thermal conversion process, and/or that is enhanced for use in bioconversion.

### BRIEF SUMMARY OF SOME OF THE PREFERRED EMBODIMENTS

These and other needs in the art are addressed in one embodiment by a method for the production of animal feedlot biomass for use as fuel in a reactor. In an embodiment, the method comprises surfacing a feedlot with a feedlot surfacing material. In addition, the method comprises collecting an animal feedlot biomass from the feedlot. Further, the method comprises preparing the collected biomass for use as fuel.

Theses and other needs in the art are addressed in another embodiment by a method for the reduction of NOₓ emissions from a coal-fired power plant. In an embodiment, the method comprises a preparing the fuel. In addition, the method comprises reducing the NOₓ emissions of the coal fired power plant by using the fuel as a reburn fuel, co-firing fuel, or both in the coal fired power plant. Preparing the fuel comprises surfacing a feedlot with a feedlot surfacing material and collecting an animal feedlot biomass from the feedlot.

Theses and other needs in the art are addressed in another embodiment by a method for the reduction of the emission of at least one heavy metal from a carbonaceous feed combustion process. In an embodiment, the method comprises preparing a fuel. In addition, the method comprises reducing the emission of the at least one heavy metal by co-firing the fuel with carbonaceous feed, using the fuel as reburn fuel in the combustion process, or both. Preparing the fuel comprises surfacing a feedlot with a feedlot surfacing material and collecting an animal feedlot biomass from the feedlot.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 illustrates an embodiment of a method for producing advanced feedlot biomass according to the principles described herein;

FIG. 2 is a bar graph comparing the higher heating value of cattle feedlot ration samples (day 1), raw manure (day 1), partially composted manure (day 32), finished composted manure (day 125), bin-stored finished compost (204-326 days), and Wyoming coal;

FIG. 3 is a bar graph comparing the proximate analyses of the equine stall cleanings (SC), cattle manure (CM), and cattle manure plus hay (CMH) on Day 0;

FIG. 4 is a bar graph comparing the proximate analyses of the SC, CM, and CMH on Day 90;

FIG. 5 is a bar graph comparing the proximate analyses of the SC, CM, and CMH on Day 180;

FIG. 6 is a bar graph comparing heating values for the SC, CM, and CMH on Day 0;

FIG. 7 is a bar graph comparing heating values for the SC, CM, and CMH on Day 90;

FIG. 8 is a bar graph comparing heating values for the SC, CM, and CMH on Day 180;

FIG. 9 is a bar graph comparing ultimate analyses for the SC, CM, and CMH on Day 0;

FIG. 10 is a bar graph comparing ultimate analyses for the SC, CM, and CMH on Day 0;

FIG. 11 is a bar graph comparing ultimate analyses for the SC, CM, and CMH on Day 90;

FIG. 12 is a bar graph comparing ultimate analyses for the SC, CM, and CMH on Day 90;

FIG. 13 is a bar graph comparing ultimate analyses for the SC, CM, and CMH on Day 180;

FIG. 14 is a bar graph comparing ultimate analyses for the SC, CM, and CMH on Day 180;

FIG. 15 is a bar graph comparing hydrogen to carbon ratio for SC, CM, and CMH on Days 0, 90, and 180;

FIG. 16 is a bar graph comparing nitrogen to carbon ratio for SC, CM, and CMH on Days 0, 90, and 180;

FIG. 17 is a bar graph comparing oxygen to carbon ratio for SC, CM, and CMH on Days 0, 90, and 180;

FIG. 18 is a bar graph comparing sulphur to carbon ratio for SC, CM, and CMH on Days 0, 90, and 180; and

FIG. 19 is a bar graph comparing phosphorus to carbon ratio for SC, CM, and CMH on Days 0, 90, and 180.

### DETAILED DESCRIPTION OF SOME OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices and connections.

Herein disclosed are methods for producing feedlot biomass for use as a fuel. Embodiments of the disclosed methods offer the potential for feedlot biomass with enhanced chemical and physical properties for thermochemical conversion, for example, pyrolysis, gasification, and combustion, including, but not limited to co-firing with coal in a coal:manure fuel blend in combustion units, use as reburn fuel for reduction of NOx, combustion or gasification in a fluidized bed system, or combinations thereof. Although the following discussion is presented in terms of cattle manure, the feedlot biomass may include, without limitation, other animal manure, including but not limited to bovine manure, porcine manure, equine manure, avian manure, and combinations thereof. Moreover, along with animal manure, the biomass may comprise other biomass such as, for example, animal carcass, crop residue, and combinations thereof. Such enhanced properties include, without limitation, maximized higher heating value (HHV), minimized ash content, minimized content of contaminant minerals that can contribute to ash agglomerization or slagging in thermochemical conversion processes (e.g. S, Cl, Na, K, P, etc), or combinations thereof. As detailed hereinbelow, improved properties are attained through various combinations of the following technologies: use of feedlot surfacing, employment of improved manure collection practices, and/or adjustment of cattle ration including, but not limited to, reduction in phosphorus content or other specified minerals which are undesirable from the standpoint of combustion, reburn, or thermal conversion.

The disclosed methods comprise several steps that, when combined, synergistically offer the potential for improved thermal conversion properties in solid feedlot manure from animal feedlots. The enhanced manure produced via the disclosed methods is herein termed “thermally-advanced feedlot biomass” (TAFB), to indicate improved thermal performance. The steps of the methods, when performed collectively, offer the potential for near-optimal levels of many biomass constituents and parameters. For enhanced performance as a fuel, it is desirable to maximize higher heating value (HHV, measured in BTU/lb or KJ/kg), fixed carbon (% or ppm), and volatile matter (% or ppm) of the TAFB, while minimizing ash/soil level (% dry basis (d.b.)) and levels of undesirable minerals including, but not limited to, phosphorus (% d.b. or ppm), sodium (% d.b. or ppm) and chloride (% d.b. or ppm). Additionally, it is desirable to maintain moisture levels at low or intermediate levels (% wet basis (w.b.)). Higher nitrogen content as urea or other organic form may be retained via the disclosed methods, and this may be desirable when TAFB is used as co-firing or reburn fuel as discussed further in Example 6 hereinbelow.

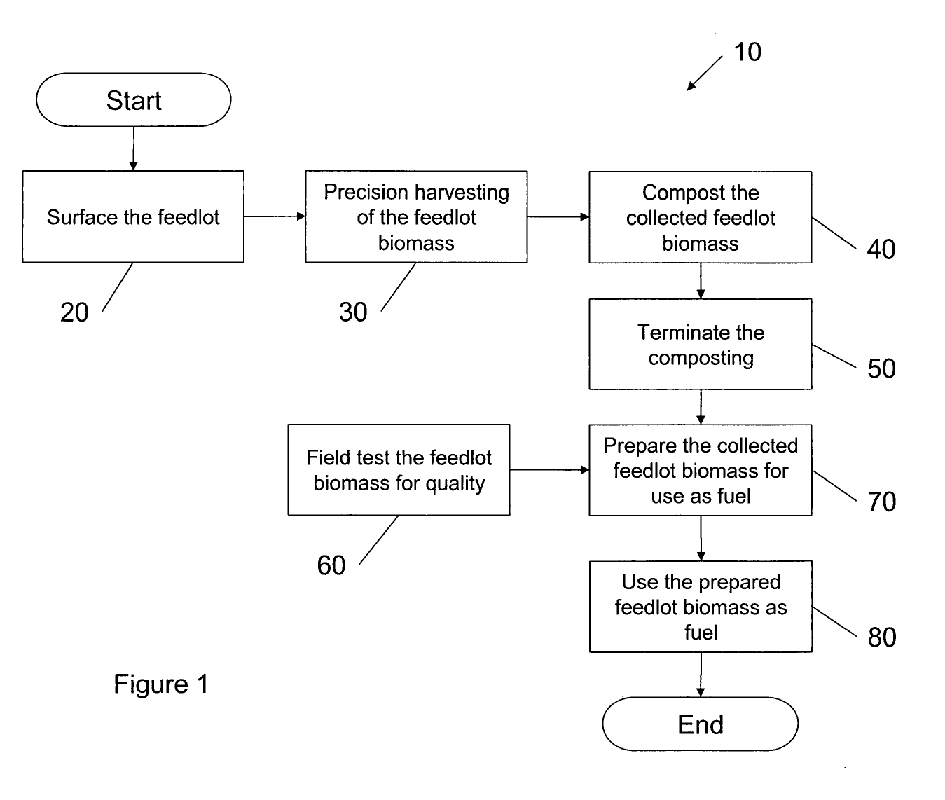

Referring now to FIG. 1, an embodiment of a method 10 for producing feedlot biomass for use as a fuel is shown. Method 10 comprises surfacing the feedlot (block 20), precision harvesting or collecting the feedlot biomass from the surfaced feedlot (block 30), composting the collected feedlot biomass (Block 40), terminating the composting (block 50), field testing the feedlot biomass for quality (block 60), preparing collected feedlot biomass for use a fuel (block 70), and using the collected and prepared feedlot biomass as fuel (block 80). Each of these blocks or subprocesses of method 10 are described in more detail below.

### Surfacing the Feedlot

Feedlot biomass (e.g., cattle manure) typically contains moisture and non-combustible ash (e.g., soil), which do not contribute to HHV, but undesirably add to total mass and loss of appreciable heat via flue gases. In general, a higher ash content is correlated with lower heating value. For example, Table 1 shows predicted higher heating value (HHV) variation as a function of ash and moisture. For calculation of the values in Table 1, feedlot manure is assumed to have an HHV of 8,500 BTU/lb on a dry ash free basis (DAF), and the HHV values were obtained as follows:

HHV BTU/lb=8,500 BTU/lb DAF×((100%−moisture, % w.b.)×(100−ash, % d.b.)/10,000)

Therefore, assuming feedlot manure has an HHV of 8,500 BTU/lb on a dry ash-free (DAF) basis, if, for example, an as-received design parameter of 2,700 BTU/lb is desired, ash contents of up to 60% d.b. would be permissible with moisture contents of 20% w.b. or less. For moisture contents of 50%, ash contents of 30% or less meet a criterion of 2,700 BTU/lb.

2,700 BTU/lb (as received)≈8,500 BTU/lb DAF×((100%−20% w.b.)×(100%−60% d.b.)/10,000)

In general, the ash and moisture content in fuel have a large effect on the flame temperature and other combustion parameters and results. Typically, decreased ash content and decreased moisture are preferred for combustion processes. In addition, higher ash content may also lead to undesirable fouling of the reactor in which the fuel is ultimately thermally converted. Higher ash percentage and particularly higher alkaline oxides can cause problems in boiler burners by causing fouling and boiler tube corrosion, and reducing the adiabatic flame temperature. High ash may also lead to high erosion rates due to abrasion. Although this can reduce the buildup of fouling deposits, it may be detrimental to tube integrity. Consequently, for purposes of combustion, reburn, or other means of thermal conversion, soil (ash) and moisture in biomass are considered contaminants. The use of low-ash manure produced via the disclosed methods offer the potential to decrease ash fouling of thermal combustion reactors.

Although some open feedlots are paved with concrete, conventional practice for manure collection and harvesting involves wheel loaders, elevating scraper, or box scraper from open, soil surface (i.e., unsurfaced feedlots) cattle feedlots. In soil surface feedlots, the underlying soil has a tendency to become entrained in the manure by cattle hooves and the manure harvesting practices. Entrainment of underlying soil increases the ash content of the manure, thereby undesirably diminishing HHV and altering nutrient concentrations in manure.

To reduce and/or prevent entrainment of underlying soil in the biomass, and thus, reduce the ash (soil) in the collected biomass, method 10 comprises the step of surfacing the feedlot 20. Surfacing the feedlot 20 may be performed by any suitable method known to one of skill in the art including, but not limited to the methods described in the Examples 1 and 2 below. The surfacing material used in step 20 may include, without limitation, concrete, fly ash, crushed bottom ash from a coal-fired power plant, or combinations thereof to produce an relatively impervious surface that reduces and/or prevents bulk mixing of soil (ash) and biomass.

In some embodiments, surfacing the feedlot 20 includes (a) placing the surfacing material on the feedlot surface; (b) hydrating, mixing, and compacting the placed surfacing material; and (c) finishing the surfacing material. The surfacing material is placed on the feedlot subgrade in multiple layers until the desired depth is achieved. During placement, the surfacing material may be consolidated with rollers capable of compacting from the bottom up. A substantially homogeneous distribution of surfacing material in each layer is preferred. Further, any spreading operations are preferably performed in such a way that zones of surfacing material of non-uniform gradation resulting from segregation in the hauling or dumping operations are reduced and/or eliminated in order to reduce and/or eliminate formation of weakened planes.

Compacting the placed material is performed to achieve a desired density. The placed material is preferably compacted to a density of greater than or equal to about 90% compaction ratio density, and more preferably greater than or equal to about 95% compaction ratio density. The moisture content of the surfacing material (e.g., fly ash) during compaction operations is maintained within a range from a determined optimum percentage to about 2 percentage points above to about 4 percentage points below the optimum percentage. Material moisture level, roller weight, and/or lift thickness may be adjusted to achieve the desired density, as is known to those of skill in the art. Prior to compaction, grading may be used to obtain the desired shape and thickness. When additional lifts or collection of the feedlot biomass are necessary, the existing layer may be lightly sprinkled with additional surfacing material prior to placing additional courses.

After the final course of the surfacing material is compacted, finishing of the placed, compacted material includes finishing to grade and section by blading and sealing with pneumatic steel drums or tire rollers to provide a dense, uniform surface and avoid the construction of compaction planes.

Surfacing the feedlot comprises covering at least a majority of the surface of the feedlot with feedlot surfacing material. The average depth of the surfacing material is preferably greater than about 3 inches and less than about 8 inches, although average depths greater than 6 inches or even 8 inches may be desirable in some applications. Various areas of the feedlot may be surfaced to a greater average depth, for example, in front of a loading chute, or other areas where high traffic and/or vehicle traffic is anticipated.

To limit absorption of precipitation, and expedite natural evaporative drying, the feedlot should be designed with good feedlot drainage to promote rapid shedding of precipitation. It is desirable to maintain the biomass in relatively dry solid form (i.e. less than about 50% moisture on a wet basis, except perhaps during or shortly after precipitation). Drier biomass normally has a lower rate of decomposition (carbohydrate conversion) and carbon loss as compared to relatively wet manure, all other conditions being equivalent.

Feedlot surfacing, for example with fly ash or crushed-bottom ash surfacing, may also have potential as a means of moisture retention or perhaps moisture-replenishment for lowering particulate matter (PM) emissions from feedyards, which are subject to the 1990 Federal Clean Air Act Amendment. In embodiments, average dust concentration is reduced by surfacing feedpens with, for example, crushed bottom ash surfacing or rototilled mixture of fly ash and caliche, or by interspersing such surfaced feedpens with unsurfaced feedpens.

### Collecting the Feedlot Biomass

Referring again to FIG. 1, in block 30, the feedlot biomass is precision collected or harvested. Conventional practice of harvesting feedlot biomass (e.g., manure) involves the relatively infrequent collection of feedlot biomass after pens of animals are shipped, Without being limited by this or any particular theory, on a dry weight basis, fiber and ash contents in feedlot biomass increase as decomposition progresses, and nitrogen losses of 40-60% can occur on the feedlot surface. For example, ash content in manure on the feedlot surface increases in time from about 15% ash/85% volatile solids (VS) for fresh manure, about 25%-30% ash/70%-75% VS at 5-6 months, to about 50% ash/50% VS at 12 months (if the feedlot surface remains wet in the interim, ash content can increase even more, and VS content decreases consistently). Since HHV varies inversely with ash content and moisture content, block 30 of method 10 comprises relatively frequent biomass collection. Higher collection frequency contributes to higher biomass quality in terms of greater retention of carbon, volatile solids, and nitrogen, and to reduced particulate (dust) emissions from open surfaces. More frequent collection may also serve to increase nitrogen by reducing undesirable NH₃ volatilization at the feedyard.

In block 30, the collection frequency according is preferably performed at least as frequently as once every 1-2 months, more preferably at least once a month, and even more preferably about once a week weekly. The frequency of collection of the feedlot biomass in block 30 may be influenced by several factors including, without limitation, the season. For example, collecting the feedlot biomass during or shortly after a relatively dry season often results in a desirable reduced moisture content. Moreover, more frequent removal of feedlot biomass also offers the potential to reduce mud, and fly problems, as well as reducing air quality issues due to the release of CH₄, H₂S, NH₃, amines, volatile organic compounds, phenols, p-cresol, esters, mercaptans, and other chemicals (which may lead to odor release) during manure storage at the feedlot.

Without being limited by this or any particular theory, the combustion-related characteristics of conventional unsurfaced feedlot manure biomass varies with vertical location within the manure pack—the best manure quality (highest HHV) is typically found near the top surface (loose material, e.g., 0-2 inches of the manure pack), intermediate manure quality is found in the middle layers (compacted layers) of the manure pack, and lower quality is found near the bottom of the manure pack. For example, the soil-manure interfacial layers may comprise 70-90% ash and 500 BTU/lb to about 1500 BTU/lb HHV with high or moderate moisture contents. Consequently, collecting the feedlot biomass according to block 30 comprises collecting the uppermost ½ to ⅔ of the manure pack.

The physical collection of the upper layers of the manure compact in block 30 is preferably precision harvested using, without limitation, wheel loader with a cleated bucket, box scraper (e.g., tractor drawn box scraper), elevating (paddle) scraper (e.g., self-propelled elevating scraper), road grader, or combinations thereof. The scrapers may be used to harvest surface manure or cut-to-grade through manure pack, and bottom cleats on wheel loader bucket may be used to elevate the cutting edge above surfacing layer, underlying soil or interfacial (soil/biomass) layer.

### Composting the Feedlot Biomass

Referring again to FIG. 1, in block 40, the harvested or collected feedlot biomass (e.g., manure) is composted. In particular, the collected feedlot biomass is partially composted to achieve initial mixing and heating to yield partially composted (PC) manure. Although the partial composting may be performed by any suitable means, one preferred method of composting utilizes at least one compost pile or windrow (elongated row) mechanically heaped drying purposes.

The duration of partial composting generally ranges from about 20 to 60 days. In embodiments, the duration of partial composting preferably ranges from 30 to 60 days and more preferably ranges from 40 to 55 days. In some embodiments, block 30 may also comprise the addition and/or maintenance of adequate moisture and turning of the feedlot biomass to initiate and promote partial composting.

The compost pile or windrow may include additional materials including, without limitation, mortality biomass (as discussed below and in Example 7), crop residues such as cotton gin residue (CGR) (as discussed in Example 3), or combinations thereof. The amount of crop residue and/or mortality biomass depends on a variety of factors including, without limitation, the type of feedlot biomass, the size of the animal carcass, etc. However, for many cases where crop residues are included in the compost pile, the compost pile preferably comprises about 0% to about 50% crop residues. For many cases where mortality biomass is included in the compost pile, the compost pile preferably comprises more than about 0.2 ft³ of feedlot biomass (e.g., manure) per pound of mortality biomass (e.g., animal carcass), and preferably between about 0.2 ft³ and 1.0 ft³ of feedlot biomass per pound of mortality biomass.

It should be appreciated that the use of mortality biomass in the compost pile also helps cattle feedlots address animal carcass disposal issues. In particular, in many cattle feedlots, horses are utilized to herd and ride pens to inspect the cattle. Death losses of perhaps 1% to 2% or more of the cattle population along with equine mortality often leads to animal carcass disposal issues. However, such mortality biomasses may be buried with the compost pile according to block 30. As discussed in Example 7 hereinbelow, the incorporation of mortality biomass into the feedlot biomass offers the potential to advantageously increase the HHV of the resulting TAFB.

### Terminating the Composting

Referring again to FIG. 1, upon completion of partial composting in block 40, the composting process may be terminated or retarded in block 50 by allowing the partially composted feedlot biomass to attain a moisture content less than 30-35%, more desirably less than or equal to about 20%, preferably less than or equal to about 10%. Alternatively, the composting process may be terminated or retarded according to block 50 by collecting the partially composted feedlot biomass at low moisture from the compost pile before it has finished composting and placing the partially composed feedlot biomass in bulk storage. The concentration of volatile solids, and thus the heating value, may be increased by reducing the moisture content of the collected manure. Therefore, dry storage without moisture penetration is preferably utilized. In general, the collected partially composted feedlot biomass may be stored by any suitable means including, without limitation, dry-stored in a greenhouse, dry-stored in a covered or uncovered windrow, dry-stored in a covered pit silo, or combinations thereof.

In some embodiments, method 10 may further comprise adjusting feed ration (animal feed), e.g. cattle ration, to alter at least one mineral or constituent in the feedlot biomass (e.g., manure) to enhance the performance of the reactor ultimately used to thermally convert the fuel. For example, the level of a potentially problematic nutrient, salt, and/or other chemical constituent in feedlot ration may be altered to alter the subsequent feedlot biomass. Lowering the levels of mineral ingredients such as phosphorus, chlorine, or sodium in the animal diet to near NRC dietary requirement levels offers the potential to reduce the amount of these non-absorbed minerals in the excreted feces or urine, while maintaining animal (e.g. cattle) performance. The level of phosphorus in the feedlot biomass may be reduced by decreasing the amount of phosphorus in the feed ration by about 33%. The amount of salt in the biomass may also be adjusted by harvesting the manure more (or less) frequently, thus controlling the amount of leaching.

The nitrogen content of the feedlot biomass may also be reduced by altering the feed ration. For example, the crude protein in the feed ration may be reduced from about 13% to about 11%, thus reducing the excreted nitrogen accordingly. In alternative embodiments, the nitrogen content of the feedlot biomass is reduced by altering the crude protein in the feed ration to less than about 10%. Reduction in nitrogen content may also decrease undesirable NH₃ volatilization at the feedlot.

In embodiments, the TAFB has a higher Ca content than conventionally produced manure. This may be due to scalping of fly ash feedlot surfacing material into the manure, Ca absorption from fly ash into the biomass, and/or reduced dilution of calcium from ash content in low ash feedlot biomass (LA-FB). Without being limited by this or any particular theory, increased Ca in a fuel reduces NOₓ and SO₂.

### Preparing Collected Manure for Use as Fuel

Referring again to FIG. 1, in block 70, the collected feedlot biomass is prepared for use as a fuel. Preparing the biomass for use as fuel may comprise air drying the collected feedlot biomass (e.g., manure). For example, air drying of the collected manure comprises drawing the partially composted (PC) manure out of bulk storage and placing it in thin beds for air drying on a concrete, fly ash, or crushed bottom ash floor (e.g., under greenhouse). The moisture content of the feedlot biomass is preferably reduced to within the range of from about 10% w.b. to about 12% w.b., more preferably reduced to less than 10% w.b., and even more preferably reduced to less than 7% w.b. In select embodiments, the moisture content of the feedlot biomass is is reduced to less than 5% w.b.

Preparing the collected feedlot biomass for use as fuel in block 70 may further comprise altering the particle size of the collected dried feedlot biomass. The particle size of the feedlot biomass may be changed in a two-stage process. The first stage comprises pre-grinding the feedlot biomass and the second stage comprises grinding or pulverization of the feedlot biomass. Moisture reduction (e.g., by greenhouse drying) may optionally be carried out between the two stages. The feedlot biomass may be pre-ground by any suitable means including, without limitation, using a hammer mill. The collected dried feedlot biomass is preferably pre-ground to a median particle size characterized by from about 42% to about 51% passing a 100 mesh (<149 micron screen). Pulverizing the collected dried feedlot biomass may be performed by any suitable means including, without limitation, by using an impact mill such as a Vortec Impact Mill®, available from Vortec Mfg. Co., Long Beach, Calif. The collected feedlot biomass is preferably pulverized in the second stage preferably to a median particle size of about 74% passing a No. 200 standard (<75 micron screen) mesh sieve, and more preferably pulverized to a median particle size of about 50% passing a 70 μm sieve.

In select embodiments, the collected dried, pre-ground, and pulverized feedlot biomass is further prepared for use as fuel in block 70 by removing ash to decrease the ash content (and concomitantly increase the heating value) of the biomass. The need for ash separation may be dictated in part by the field testing in block 60 described in more detail below. In general, removal of ash may be performed by any suitable method(s) including, without limitation, ballistic separation, mechanical separation (e.g., via shaker, screen, sieve, etc.), or combinations thereof. The Micrometric Separator available from DDS Technologies USA, Inc., Boca Raton, Fla. and described in U.S. Pat. No. 6,848,582, which is hereby incorporated herein by reference in its entirety, is particularly suited to separation and removal of ash from the feedlot biomass. Although a single pass may be used, the pulverized feedlot biomass preferably makes a plurality of passes through the separator to enhance ash removal. For multiple passes, the pulverized feedlot biomass may be recirculated through a single separator, or multiple separators may be arranged in series, with the feedlot biomass passing through the first separator, then the second separator and so on. As described in Example 9 hereinbelow, for pulverized feedlot biomass, three passes through the Micrometric Separator described in U.S. Pat. No. 6,848,582 offers the potential to reduce the ash content in the feedlot biomass by about 50% (e.g., from about 54% d.b. to about 26% d.b.). In other words, multiple passes through a Micrometric Separator offers the potential to reduce the ash content in the feedlot biomass by about half. Rather than simply discarding the removed ash, it may be sold or used as fertilizer for agricultural or horticultural crops, used as paving material for feedlots, dairies, etc., used as construction material, and other uses known to those of skill in the art.

### Field-Testing for Quality

Referring again to FIG. 1, in block 60, the feedlot biomass may be tested for quality in the field prior to preparing the feedlot biomass for use as fuel in block 70. Field testing for quality may comprise determining the bulk density of the feedlot biomass in order to predict ash content. For example, the average of ASAE and ASTM methods, may be used to determine bulk density, as described in Example 5 hereinbelow. In general, a bulk density less than about 40 lb/ft³ at 6.4% moisture is correlated with low ash feedlot biomass (LA-FB), and a bulk density of more than about 40 lb/ft³ at 5% moisture is correlated with high ash feedlot biomass (HA-FB, or conventional FB). Surfacing the feedlot and the use of precision collection of the feedlot biomass according to blocks 20, 30, respectively, may result in low ash feedlot biomass.

In addition to, or as an alternative to bulk density testing, colorimetric properties may be used to field test the biomass for quality in block 60. Typically, a light brown color correlates to a relatively low moisture biomass, a dark brown color correlates to a relatively high moisture biomass, and a grayish white or pale yellow color correlates to a relatively high caliche content.

In select embodiments, near-infrared spectroscopy (NIRS) may be used to field test the feedlot biomass for quality. As described in Example 7, NIRS may be employed to assess ash and moisture content of the feedlot biomass, which are determinants of higher heating value (HHV) and feedlot biomass quality. The NIRS may be performed by any suitable spectrometer such as the Perten Diode Array 7200 Spectrometer available from Perten Instruments of Stockholm, Sweden. It should be appreciated that the use of NIRS enables relatively quick analysis and assessment of the feedlot biomass quality.

Other types of testing including, without limitation, particle size, biomass texture, protein content, or combinations thereof may also be used to field test the feedlot biomass for quality. Sieve analysis may be used to determine sand, silt, clay, and/or gravel fractions. Further, caliche and rock particle size and content may be visually determined. Protein content may be determined by any suitable method known in the art. In general, a relatively high protein content correlates to a low ash/low moisture biomass having high HHV and increased nitrogen, while relatively low protein content correlates to a relatively high ash/high moisture biomass having low HHV and reduced nitrogen. Other constituents (e.g., P, K) may be used as a general indicator of protein content.

### Properties of the Thermally Advanced Feedlot Biomass (TAFB)

Embodiments of method 10 produce thermally advanced feedlot biomass (TAFB) having a relatively large high heating value (HHV) as compared to conventionally collected (e.g., soil surfaced feedlot) techniques. In particular, method 10 produces TAFB with a HHV between about 7800 and about 9200 BTU/lb on a dry ash free (DAF) basis, a HHV greater than 3000 BTU/lb, or even greater than 3500 BTU/lb, on a wet basis (w.b.), and a HHV greater than about 4000 BTU/lb, or even greater than 4500 to 4800 BTU/lb, on a dry basis (d.b.).

As compared to conventionally collected feedlot biomass, embodiments of method 10 produce TAFB having a higher value of at least one parameter selected from HHV, volatile matter, and fixed carbon. In particular, the TAFB produced by method 10 has a higher heating value (HHV) at least 50% greater than conventionally obtained feedlot biomass, and a DAF (dry, ash free) HHV at least 5% greater than conventionally obtained feedlot biomass, alternatively at least 10% greater than conventionally objective feedlot biomass. In addition, the TAFB produced by method 10 has a volatile solids content at least 40% greater than conventionally obtained feedlot biomass, alternatively at least 50% greater than conventionally obtained feedlot biomass, alternatively at least 55% greater than conventionally obtained feedlot biomass. Further, the TAFB produced by method 10 has a carbon content at least 50% greater than the carbon content of conventionally obtained feedlot biomass, alternatively at least 60% greater than the carbon content of conventionally obtained feedlot biomass.

In addition to the desirable increases in HHV, volatile solids, and fixed carbon, embodiments of method 10 produce TAFB with a reduce ash content. In particular, the TAFB produced by method 10 has an ash content less than about 40% wet basis (w.b.), alternatively less than 30% w.b., alternatively less than 20% w.b. The ash content on dry basis (d.b.) of the TAFB produced by embodiments of method 10 is less than about 60%, alternatively less than 40% d.b., alternatively less than 30% d.b. As compared to feedlot biomass collected using conventional (e.g., soil surfaced feedlot) techniques, the TAFB produced by embodiments of method 10 is reduced by more than 10% dry basis (d.b.), alternatively reduced by more than 20% d.b., alternatively reduced by more than 25% d.b.

### Use as Fuel

Referring again to FIG. 1, the produced TAFB may be used as a fuel according to block 80. In general, the TAFB may be used for fuel in a thermal conversion process (e.g., the TAFB may be pyrolyzed, gasified, combusted, or a combination thereof) either alone or as a component in a mixture. For example, the TAFB produced in method 10 may undergo thermal conversion within a mixture, where the addition of the TAFB improves the fuel in some way (e.g., reduces NOₓ emissions, reduces heavy metal emissions, etc.) as further described hereinbelow. Suitable fuel sources with which the TAFB may be mixed prior to thermal conversion include, without limitation, crop residues (e.g. cotton gin residue), rubber tires, wood, coal, lignite and combinations thereof The suitable fuel may be preground and/or pulverized prior to or after mixing with TAFB. In embodiments, the fuel mixture comprises a mixture of suitable fuel and TAFB in a ratio of about 80:20, alternatively, a ratio of about 90:10. In some embodiments, the suitable fuel-TAFB blend comprises from about 5 weight % to about 50 weight % TAFB. Further, in embodiments, the TAFB may serve as fuel for bioconversion.

Co-firing is defined as the firing of a renewable fuel (i.e., biomass) along with the primary fuel (i.e. coal, lignite, natural gas, furnace oil, etc.). For instance, the TAFB prepared according to embodiments of method 10 may be blended with coal for fuel. Prior to mixing the coal with the TAFB to produce the coal and feedlot biomass blend (CAFB), the coal is preferably pulverized. In embodiments, the coal is pulverized to 80% passing a No. 200 standard mesh sieve and 70% passing No. 325 mesh sieve, and is blended with pulverized TAFB to create a 90:10 weight percent coal:feedlot biomass blend. In alternative embodiments, coal is blended with pulverized TAFB to yield a 80:20 weight percent coal:feedlot biomass blend. In some embodiments, the coal:feedlot biomass blend comprises from about 5 weight % to about 50 weight % TAFB. In general, a coal and feedlot biomass blend (CAFB) may be used to fuel any suitable combustion unit including, without limitation, an an updraft-fired combustion unit, a downdraft-fired combustion unit, etc. A CAFB blend comprising pre-ground and then pulverized Wyoming Powder River Basin coal and partially composted (PC) feedlot manure as a 90:10 weight percent coal:manure blend is characterized in Table 2. As discussed hereinbelow, NO emissions for a coal:FB blend may be reduced relative to coal alone even with the higher nitrogen content of biomass than coal.

NOₓ is produced when fuel is burned with air. The N in NOₓ can come from the N containing fuel compounds (e.g., coal, biomass) and from the nitrogen in the air. NOₓ generated from fuel is known as fuel NOₓ, while NOₓ formed from air is referred to as thermal NOₓ. NOₓ and volatile organic compounds (VOCs) released into the atmosphere, react in the presence of sunlight and produce ozone or smog which may be damaging to health. It has thus been mandated that NOₓ, a smog precursor, be reduced. One method of reducing NOₓ is reburning, where additional fuel (e.g. coal or natural gas) is injected downstream from the primary combustion zone to create a fuel-rich zone where NOₓ is reduced through reactions with the hydrocarbons. After reburn, a burnout zone may be used to complete the combustion process, as is known to those of skill in the art. While the main goal of co-firing is to produce power, the main objective of the reburn process is to reduce NO to harmless N₂, not the production of power.

Pulverized TAFB maybe used as reburn fuel. In embodiments, no major equipment modification is required for combustion of TAFB in coal fired furnaces. The use of TAFB as reburn fuel may be desirable due to the reduced cost/increased availability of FB as compared with coal. As discussed in Example 6 hereinbelow, the use of TAFB as reburn fuel may result in more effective reduction of NOₓ than conventional reburn fuel (e.g., coal, natural gas) or conventional high ash feedlot biomass. Reduced ash in the manure may result in more effective reduction of NO emissions during the combustion of coal. In particular, paving the feedlot may yield a biomass having higher nitrogen in low ash (LA) manure, potentially because of the lower dilution with soil (ash). Without being limited by this or any particular theory, it is believed that the nitrogen in TAFB exists as urea or other organic forms that decompose to NH₃ during pyrolysis which is capable of reducing NO produced by coal during co-firing or reburn as discussed in Example 6 hereinbelow. In embodiments, the level of NO is reduced by more than 10% upon co-firing of a coal:TAFB blend when compared to firing of coal in the absence of biomass, alternatively the NO is reduced by more than 20% upon co-firing of a coal:TAFB blend when compared to firing of coal in the absence of biomass, alternatively the NO is reduced by more than 30% upon co-firing of a coal:TAFB blend when compared to firing of coal in the absence of biomass. Further, use of TAFB as reburn fuel reduces heavy metal emissions and mercury emissions in the flue gas.

Upon combustion of TAFB in coal combustion systems, the ash (e.g., fly ash, bottom ash, cyclone ash, etc.) in the reactor (e.g., coal-fired boiler) may be removed and used to advantage. In embodiments, ash from the reactor comprises about 20% bottom ash and about 80% fly ash. For example, the ash may be used for paving of roads, for paving feedyards, or a combination thereof. When used as a constituent of paving material, the fly ash or bottom ash from the reactor may be crushed, mixed, partially hydrated, or a combination thereof prior to use.

The higher capital cost of feedlot paving and marginally higher cost of increased frequency of manure collection according to embodiments described herein may be economical when TAFB biomass is used as reburn, co-firing, gasification, or bioconversion biofuel. Environmental ramifications of using mass quantities of manure as fertilizer (and concomitant potential for water eutrophication), the higher cost of alternative reburn fuels compared to TAFB, as well as the value added by the potential NOₓ or other constituent emissions reduction (e.g., mercury) should be considered.

### Examples

The following examples are given as particular aspects of the embodiments described herein and to demonstrate the practice and advantages thereof. It is understood that the examples are given by way of illustration and are not intended to limit the specification of the claims to follow in any manner.

### Example 1

### Feedlot Surface Treatment-Fly Ash or Crushed Bottom Ash

Embodiments described herein may comprise surfacing a feedlot to reduce soil (ash) entrainment in the feedlot biomass. One technique for surfacing a feedlot comprises the placement of hopper fly ash or a compacted layer of crushed bottom ash. This technique was investigated using crushed bottom ash from a coal-fired power plant fired with Wyoming Powder River Basin Coal in several feedpens of a 30,000-head beef cattle feedlot.

The system was then evaluated using several surfaced and unsurfaced feeding pens to determine (a) quality of the resulting surface following utilization by cattle and manure harvesting by machinery and (b) feedlot manure characteristics relative to adjacent or nearby conventional unpaved feedpens. Results of replicated experiments comparing characteristics of cattle feedlot manure harvested from conventional unsurfaced feedpens (control pens) versus feedpens surfaced with 3-8 inches depth of compacted crushed bottom ash (crushed ash pens) are reported in Table 3. Feedpens surfaced with a rototilled mixture of native caliche soil and hopper fly ash were also compared but this surface did not prove viable.

The data in Table 3 show that surfacing the feedpens with crushed bottom ash from a coal fired power plant fueled by Wyoming coal significantly reduced harvested manure ash content (39.25% vs. 65.71% ash dry basis (d.b.)); increased HHV (3,520 vs. 1,982 BTU/lb w.b.; and 4,842 vs. 2,601 BTU/lbs d.b.); increased carbon (26.21% vs. 15.78% d.b.); and increased moisture slightly (27.98 vs. 23.10% w.b.) as compared to manure from adjacent pens with soil-surfacing only.⁷ The manure from crushed ash surfaced pens was also superior in quality to manure collected from pens with compacted admixture of caliche soil and crushed hopper fly ash. As shown in Table 4 which shows nutrient analysis of cattle feedlot manure, data also compared favorably with cattle feedlot manure collected from typical soil surfaced feedpens on a nutrient basis (combustion properties not determined). In Table 4, w.b. is as-received or wet basis. d.b. is dry basis.

### Example 2

### Feedlot Surface Treatment-Fly Ash

The effects of feedlot surfacing materials (soil vs. coal-ash paved) and partial composting on feedlot biomass (FB) characteristics for use in thermochemical conversion involving reburn or co-firing with coal or lignite were investigated. Feedlot biomass was harvested from 12 fly ash-paved pens and 6 soil-surfaced pens and was windrow-composted. Table 5 shows an analysis of the fly ash from Southwestern Public Service Company (XCEL Energy) that was used to pave the feedlot as described hereinabove.

Replicated experiments were used to compare characteristics of cattle feedlot manure harvested from conventional unsurfaced feedpens (control pens) versus feedpens surfaced with 6-8 inches depth of hydrated compacted fly ash from a coal-fired power plant.

Table 6 presents proximate and ultimate analyses of as-collected (un-composted) FB harvested from soil-surfaced cattle feedpens and crushed fly ash (FA) feedpens. The data in Table 6 show that surfacing the feedpens with fly ash from a coal fired power plant fueled by Wyoming coal significantly reduced harvested manure ash content. The ash content dry matter basis was 66% lower for FB from the paved (20.20%) vs. the un-paved pens (58.73%). Moisture content was similar for the as-collected HA-FB and LA-FB 19.81 vs. 20.27% w.b. (˜20% w.b.) prior to composting, as shown in Table 6. Consequently, HHV was lower (about half) for the HA-FB than for LA-FB, both on as-received basis (2,710±34 vs. 5,764±147 BTU/lb w.b.) and dry basis (3,380±14 vs. 7,229±92 BTU/lb d.b.). The LA-FB showed about 10% higher HHV on a dry ash free (DAF) basis as compared to HA-FB (9,059±13 vs. 8,200±327 BTU/lb DAF). On a dry matter basis, volatile matter of HA-FB was 33.8% as compared to 64.6% for LA-FB, while fixed carbon was 7.5% for HA-FB and 15.2% for LA-FB.

As shown in Table 6, data also compared favorably with cattle feedlot manure collected from typical soil surfaced feedpens on a nutrient basis. LA-FB contained about twice the total carbon and hydrogen as HA-FB, and about 50% higher N (3.11% vs. 1.94% for LA-FB and HA-FB respectively) and S (0.67% vs. 0.42% for LA-FB and HA-FB respectively). However, expressed on an energy basis (lbs S per million BTU), sulfur content was lower in the LA-FB. Chlorine content of the manure was essentially the same for both HA-FB and LA-FB (average of 0.376% d.b.).

Elemental analysis of ash residue and trace minerals (S, P, Cl, Na, metals etc.) for the uncomposted LA-FB and HA-FB are presented in Table 7. Data in Table 7 represents one composite (n=1) of three samples of each FB material, or of lignite, or coal. FB, TXL, and PRB coal were calcined at 1100° F. (600° C.) prior to analysis. Differences in elemental composition of sample-ash varied depending on the type of feedlot surfacing material. Compared to HA-FB, the LA-FB contained lower Si, Al, Fe and Ti, but was higher in Ca, Mg, Na, K, P, S, Cl, and Ba. These results are based on one composite sample per FB type, and should be interpreted with caution.

### Example 3

### Composting/Bin Storage of FB from Un-Surfaced Feedlots

The effects of composting with and without crop residues followed by in-bin storage and thin-bed drying in a greenhouse, followed by pre-grinding and pulverization on the combustion characteristics of feedlot biomass from soil surfaced (i.e., unsurfaced) feedlots were investigated. A windrow composting of manure from a conventional soil-surfaced feedlot for 32 and 125 days was compared to no composting (1-day). Comparison of the proximate and elemental analyses of composted feedlot manure after 1, 32, and 125 days of composting are presented as Table 8. Comparison of the ultimate analysis of composted feedlot manure after 1, 32, and 125 days of windrow composting are presented as Table 9. Subsequent in-bin storage for 6-11 months duration for uncomposted/raw manure (RM), partially composted (PC, 32 days) manure, and finished compost (FC, 125 days) further reduced combustion fuel properties as shown in Table 10 which is a manure analysis summary for uncomposted, partially composted (PC), and finished compost (FC) following 204, 297, and 328 days of bin storage under roof.

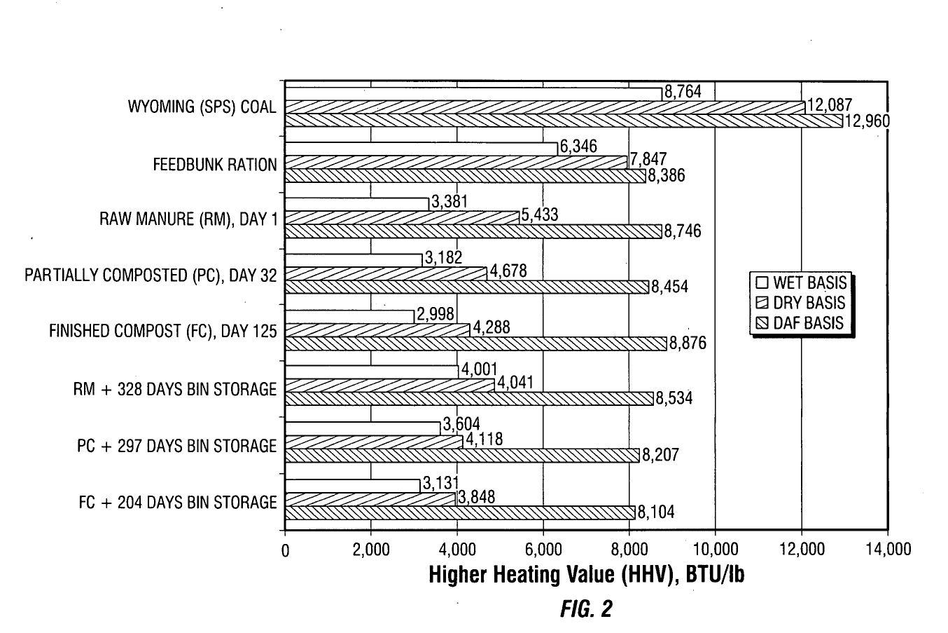

FIG. 2 is a bar diagram comparing the HHV from feedlot cattle ration samples (day 1), raw manure (RM, day 1), PC manure (day 32), FC (day 125), and Wyoming coal. HHV of a typical Wyoming coal sample is included in FIG. 2 for comparison. Manure data are averages of composting windrows with and without crop residues added at less than 5% by volume. As seen in FIG. 2, the RM, PC, and FC manure averaged across manure composted with and without crop residue additives were comparable in higher heating value on a dry-ash free (DAF) basis to cattle ration collected from feedbunks. HHV of RM, PC, and FC was further reduced during bin storage for 204-328 days.

### Example 4

### Composting/Bin Storage-Fly Ash Surfaced Lots vs. Soil Surfaced Lots

LA-FB and HA-FB from Example 2 was partially composted in windrows for 55 days and 51 days, respectively. Proximate and ultimate analyses of partially composted (PC) LA-FB-PC and HA-FB-PC are presented in Table 11. Proximate analysis showed that both PC materials had similar moisture content 17.0% w.b. and 19.6% w.b. for HA-FB-PC and LA-FB-PC, respectively. On a dry basis, the LA-FB-PC had ⅓ the ash (20.5% vs. 64.9% for LA-FB-PC and HA-FB-PC respectively), twice the volatiles, and more than three times the fixed carbon as HA-FB-PC. LA-FB-PC had 164% higher HHV as HA-FB-PC (d.b.) and 16% higher HHV on a dry ash-free (DAF) basis as HA-FB-PC (average 8,931 BTU/lb for LA-FB-PC and 7,682 BTU/lb for HA-FB-PC). Ultimate analysis showed that LA-FB-PC had over twice the total carbon and hydrogen as HA-FB-PC, which contribute to heating value, but also nearly twice the oxygen which suppresses HHV. LA-FB-PC contained 80% more nitrogen that HA-FB-PC, improving its usefulness for reburn fuel applications, as discussed in Example 6 hereinbelow. LA-FB-PC had 68% more sulfur than HA-FB-PC. LA-FB-PC had more than twice the Cl than HA-FB-PC and 74% higher phosphorus. On a heating value basis, LA-FB-PC had only ⅛ the ash and ⅔ the SO₂ as HA-FB-PC.

Elemental analysis of ash residue and trace minerals (S, P, Cl, Na, metals etc.) for the partially composted LA-FB and HA-FB are presented in Table 7. Compared to HA-FB, as shown in Table 7, sample ash from LA-FB contained ⅔ less silica and less than half the Al and Ti, and about half the Fe, without or with partial composting. However, LA-FB contained 2-3 times the Ca, Mg, Na, K and S than HA-FB. LA-FB-PC showed similar trends relative to HA-FB-PC, and was nearly five times higher in P and an order of magnitude higher in Cl. However, metals appeared to be similar, with HA-FB-PC slightly higher in As and Pb, and lower in Cd and Cr compared to LA-FB-PC.

Table 12 summarizes the dry basis comparison of un-composted and partially-composted FB from soil-surfaced and fly ash pens. Partial composting for 51 or 55 days increased ash and further reduced volatile matter, fixed carbon, total carbon, hydrogen and nitrogen in both HA-FB-PC and LA-FB-PC, compared to un-composted FB sources. Partial composting reduced HHV by 20% in HA-FB-PC and 2% in LA-FB-PC. Sulfur content changed slightly with partial composting, while Cl content increased in the LA-FB-PC. Results did not indicate major differences in elemental composition of sample-ash for either HA-FB-PC or LA-FB-PC resulting from partial composting.

For comparison, samples of Texas lignite (TXL) and Wyoming Powder River Basin (PBR) coal were analyzed in a similar manner as the FB materials. Table 13 shows proximate and ultimate analyses of TXL and PRB coal; moisture contents were 38.34±0.34% w.b. and 32.88±0.36% w.b., respectively, which is higher than for the FB materials of Tables 6 and 11. Ash contents were lower for the coal 8.40±3.11% d.b. vs. 18.59±0.85% d.b. for TXL. The latter value is only slightly lower than ash content for LA-FB and LA-FB-PC. Sulfur was higher (0.98±0.15% d.b.) in TXL than for PRB coal (0.41±0.03% d.b.) or either of the FB sources. On a dry matter basis, total carbon was much higher for TXL and PRB coal (60.30±0.92% and 69.32±2.82% d.b., respectively) than for LA-FB, LA-FB-PC, and HA-FB-PC or HA-FB. Nitrogen was slightly lower and phosphorus and chlorine much lower for either TXL or PRB coal compared to LA-FB or HA-FB. Compared to feedlot biomass, HHV was considerably higher for both TXL and PRB coal on an as-received basis (6,143±127 BTU/lb w.b. and 7,823±282 BTU/lb w.b.); dry basis (9,962±170 and 11,657±455 BTU/lb d.b.); and DAF basis (12,236±84 vs. 12,724±97 BTU/lb DAF).

Elemental ash analyses for TXL and PRB coal are presented in Table 7 for comparison with the FB results. Elemental ash analyses appeared more similar for TXL and PRB coal, than for the FB samples seen in Table 7.

### Example 5

### Bulk Density

Following the initial bulk sampling of harvested manure from the feedpens previously discussed in Examples 2 and 4 hereinabove, the bulk density of material in both windrows was determined. Bulk density was determined by two alternative standard methods: ASAE standard S269.4 and ASTM standard D1895B summarized as follows. ASAE standards method 5269.4 was modified slightly by utilizing a 0.028 m³ (1 ft³) wood container with inside dimensions of 30.5 cm×30.5 cm×30.5 cm rather than a 0.057 m³ (2 ft³) specified container size. The ASAE standard requires the material to be poured from a height of 61 cm (2 ft) until the container is filled. Once the container is filled, all excess material is scraped off with a straight edge level with the top of the container to establish a 1 ft³ struck volume of material. The material was then dropped 5 times from a height of 15.24 cm (6 in). Each time the container was dropped, FB would settle. Additional FB was added to the container and struck level with the surface and then the process was repeated. The manure was weighed after the fifth drop and addition of FB. This test was repeated three times with random samples each of the HA-FB and LA-FB. Three samples each of the LA-FB and HA-FB were taken to determine gravimetric moisture content after 24 hours at 75° C. in a drying oven.

The ASTM standard D 1895B required the material to be compacted in a known volume. The material was poured from a height of 61 cm (2 ft) until the container was filled. Once the container was filled, all excess material was scraped off with a straight edge level with the top of the container, and the container then weighed. This test was repeated three times with random samples of the LA-FB and three times with random samples of the HA-FB. Three samples of the LA-FB and three samples of the HA-FB were taken to determine moisture content, which was determined gravimetrically after drying for 24 hours at 75° C. in a drying oven.

Results were compared for unpaved vs. paved feedlot surface and for un-composted vs. partially composted FB. Bulk densities were determined for the un-composted FB, which showed major differences for the surfaced vs. non-surfaced pens. LA-FB from paved feedlots had a bulk density two-thirds that of HA-FB from un-paved/soil-surfaced feedlots, averaging 29 lbs/ft³ vs. 44 lbs/ft³ depending on methods used. Specifically, bulk density of LA-FB (at a moisture content of 6.40±0.24% w.b.) averaged 31.97±0.29 lbs/ft³. using the modified ASAE standard and 26.81±0.03 lbs/ft³. using the ASTM standard. By contrast, HA-FB (at 4.95±0.02% moisture w.b.) exhibited bulk densities of 46.65±0.86 lbs/ft³. with the modified ASAE standard and 40.61±0.71 lbs/ft³. with the ASTM standard. The packed FB materials (5 drops from 6 inches and refills) resulting from the modified ASAE standard exceeded that of the unpacked FB material from the ASTM method by approximately 19% and 15%, respectively, for LA-FB and HA-FB.

### Example 6

### Co-Firing/Reburn

The use of conventional high ash FB as reburn fuel was investigated. A reduction of 70-80% in NOₓ was achieved via reburning with high ash conventional FB (from 600 ppm initial level). A reduction of 10-40% was achieved using 100% coal reburn, depending on the equivalence ratio. The reduction of NOₓ determined for 50:50 weight percent coal:manure and 90:10 weight percent coal:manure blends fell in between the behavior of the pure coal reburn and the pure FB reburn. It was found that the biomass was more effective in reburning than coal. This was unexpected, as FB is higher in nitrogen than coal. Without wishing to be limited by theory, it is believed that the greater effectiveness of the biomass in reburning is due to its high volatile content (with little fixed carbon) and the release of fuel nitrogen as NH₃. The FB may release more volatiles at a faster rate than coal, and thus more rapidly produce fuel-rich areas where NO is reduced. The use of TAFB as reburn fuel rather than conventionally obtained FB may be desirable because the reduced ash content of TAFB may decrease reactor ash fouling compared with FB.

In addition, a process for handling and grinding low and high phosphorus feedlot manure (where the manure was produced from high and low phosphorus feedlot cattle ration in a feeding trial) from crushed bottom ash surfaced feedlot (i.e., LA-FB or TAFB), wherein the harvested manure was partially composted, air dried to <10% moisture, and mechanically sieved on a 100 mesh (<149 micron) screen was investigated. The prepared low and high P manure (42.6%±0.3% and 50.8%±1.2% passing 100 a mesh screen, respectively) was subsequently used in laboratory small scale pilot plant (30 kW) co-firing tests to study combustion characteristics. It was reported that co-firing of low ash/high phosphorus biomass fuel yielded lower NOₓ when compared to low ash/low phosphorus fuel for slightly rich mixture. Without being limited by this or any particular theory, P or ash composition including but not limited to Na and K might play a catalytic role on NOₓ reduction in co-firing.

A sample page of raw data from logs kept of some of the pilot scale reburn experiments in Chronological Event Reports, U.S. Department of Energy, National Energy Technology Center, Pittsburgh, Pa., in May 20-23, 2002 is shown in Table 14. Ratios of NOₓ emissions from the reburn chamber (fired with 100% PC biomass from soil-surfaced feedlot) showed a reduction of 73% NOₓ ppm basis and 75% mass basis. Subsequent experiments with optimized performance parameters (not shown) showed more than 90% NOₓ reduction.

### Example 7

### Composted Equine Mortality Biomass

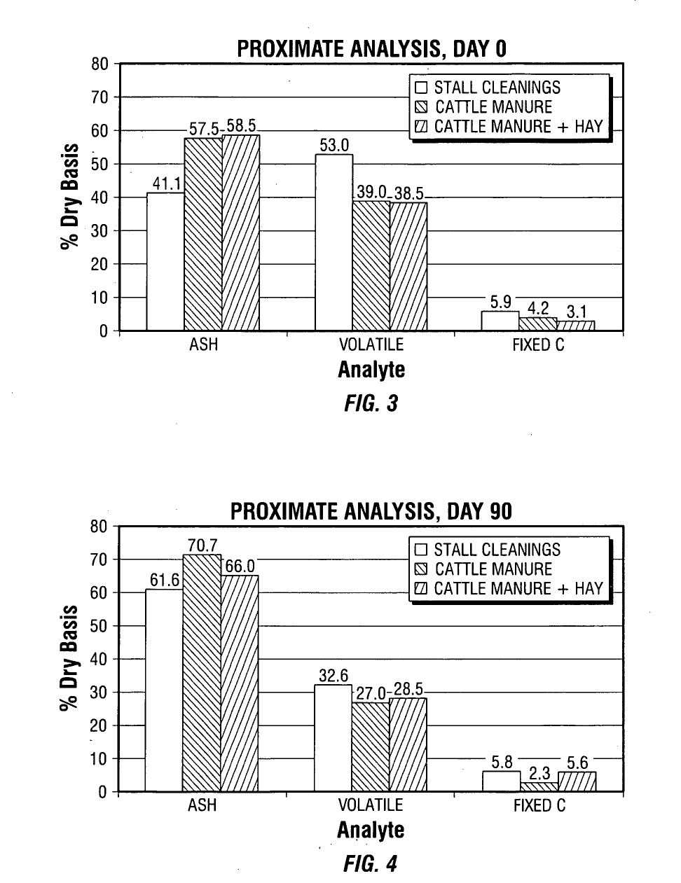

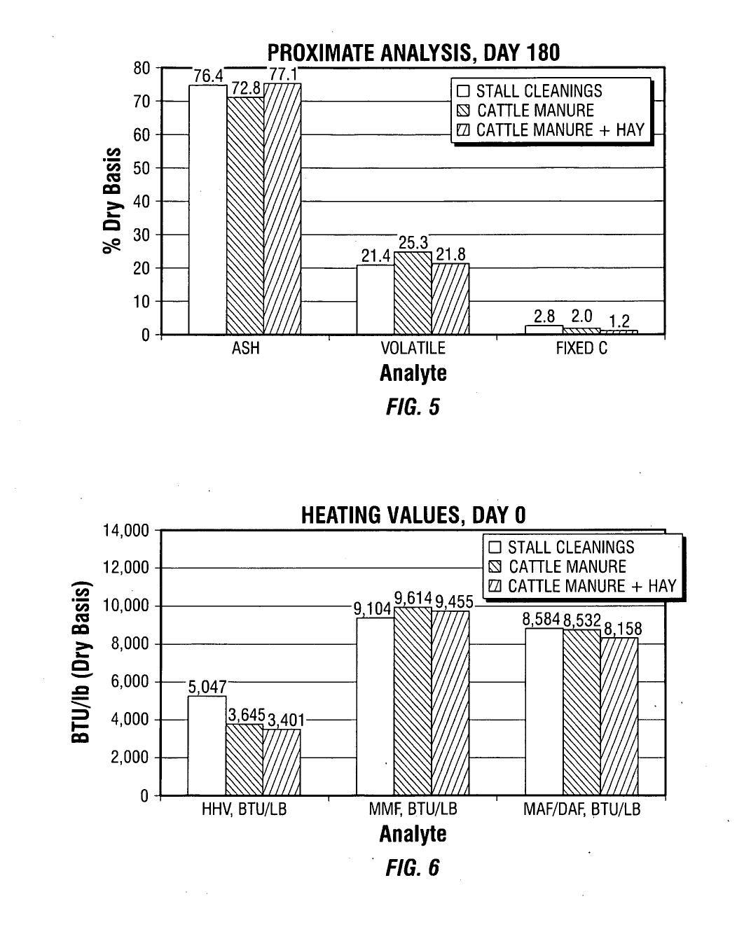

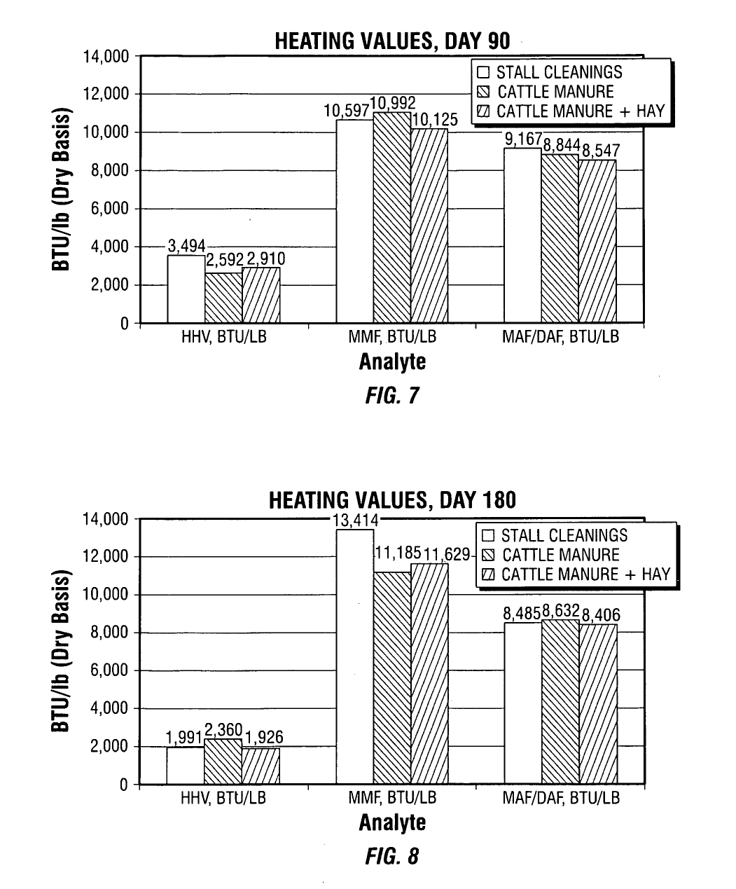

Experiments were performed in which an equine carcass was buried to approximately a 1 foot depth in the feedlot biomass. Three FBs were studied, one comprising stall cleanings (SC) comprising wood shavings and horse manure, a second comprising cattle manure (CM), and a third comprising cattle manure and hay (CMH). No turning of the windrows was performed for the first 90 days, to allow for carcass decomposition and compost pile homogenization. Samples of the FB were acquired on days 0, 90, and 180. Samples of whole carcass compost at Day 0 reflect only the feedstock (a.k.a. the manure FB) in which the carcass is to be composted, not the combined bulk of the manure FB and the carcass (as homogenization has not yet occurred). FIGS. 3 through 5 are bar graphs of the proximate analyses of the SC, CM, and CMH on days 0, 90, and 180 respectively. Ash content increases with time, while volatiles and fixed carbon decrease with time. FIGS. 6 through 8 are bar graphs of heating values for the SC, CM, and CMH on days 0, 90, and 180 respectively. MMF is the mineral-matter free heating value, which is mathematically calculated by removing sulfur and ash. DAF (MAF) is moisture and ash free and is calculated on a dry basis with ash subtracted out. For the purposes of this disclosure, the terms ‘DAF’ and ‘MAF’ are interchangeable.

Without being limited by this or any particular theory, the short-term increase in fuel value of a carcass-compost matrix is hypothesized to result from gradual release of fatty tissues into the bulk of the pile as the carcass physically degrades (call this Phase I). Livestock carcasses, including, but not limited to, beef, swine, equine, and dairy animals, may consist of as much as 25% fat on a dry matter (DM) basis. In general, fats contain more than twice the caloric potential energy as an equivalent mass of either carbohydrate or protein, which are the predominant macronutrients in the feedstock. Fats are substantially slower to degrade than sugars, starches and cellulosic materials. After a period of time, the population and total metabolic output of lipophilic organisms responsible for digesting the fats have converted that increment of fat-tissue energy into heat, reducing the bulk heating value of the pile to the initial value of the feedstock (call this Phase II). The rate of physical incorporation of the whole carcass into a compost matrix is related to the surface area per unit mass of carcass and, consequently, inversely related to carcass mass.

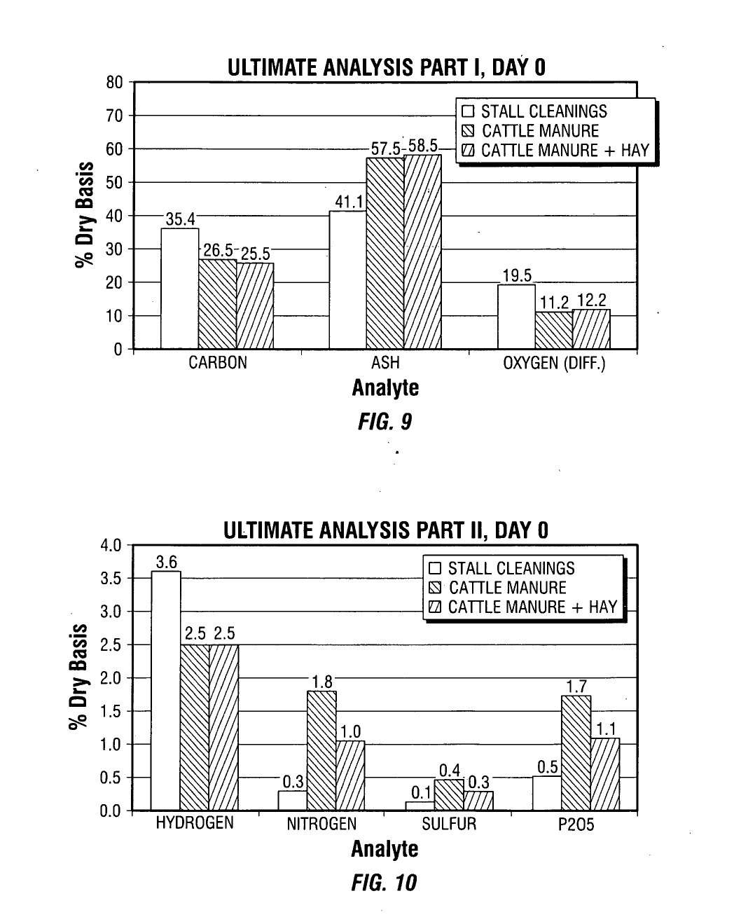

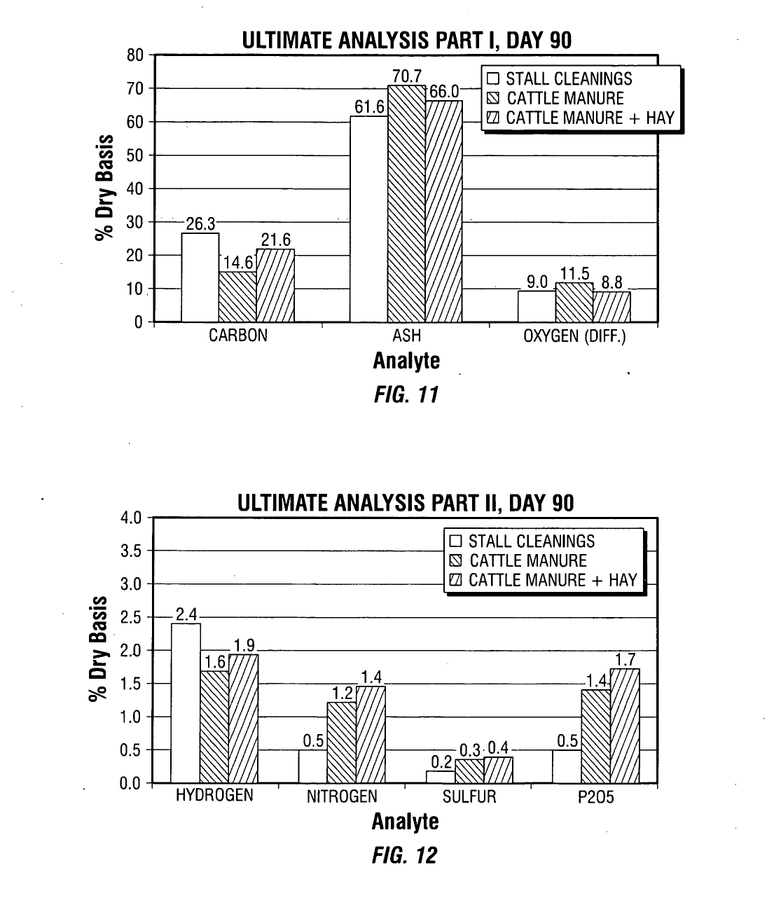

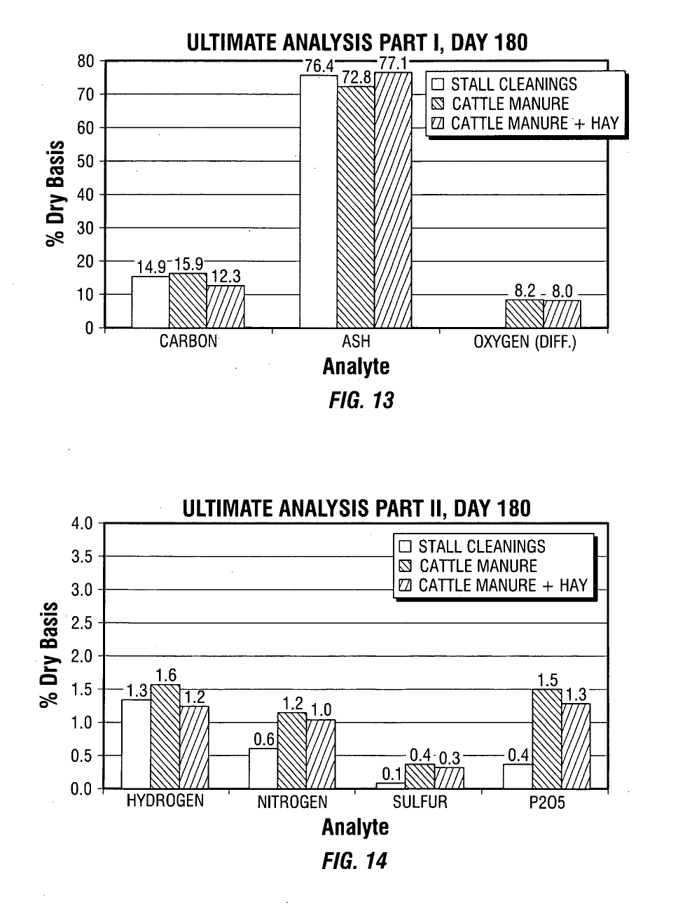

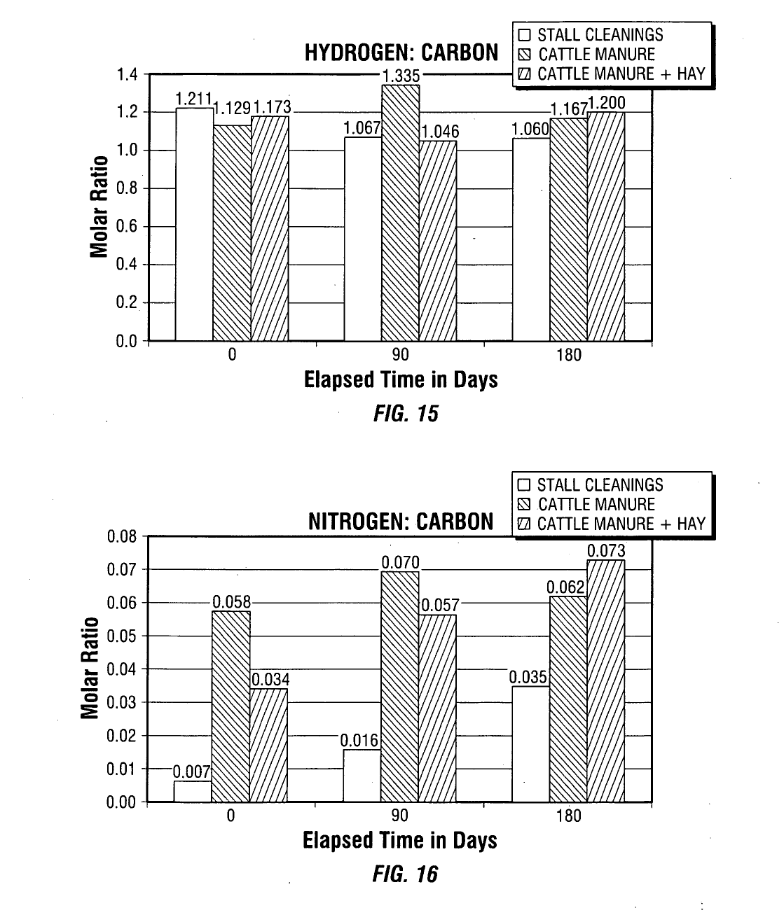

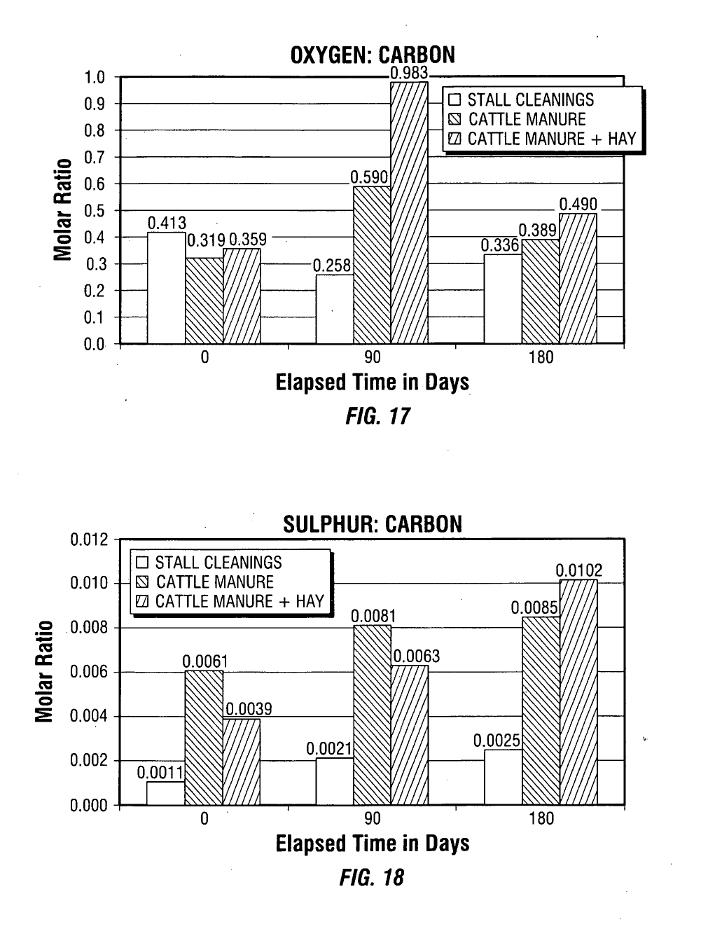

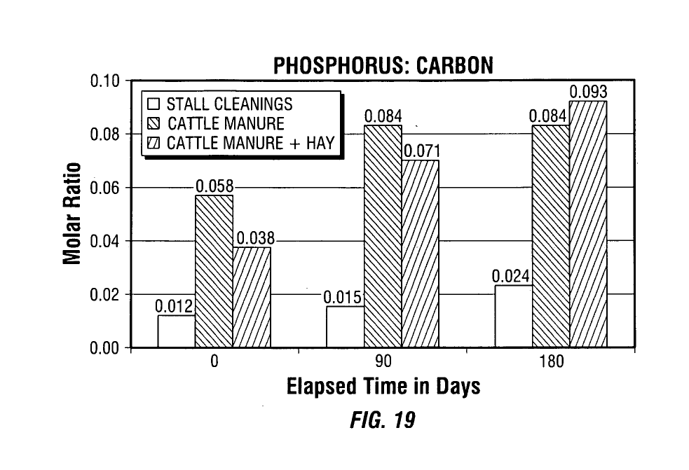

In embodiments, the FB comprises mortality biomass (MB) and the fuel value of the composted feedstock is optimized by partially composting added carcasses within Phase I. In this way, the passive composting process is ceased and the fat-enriched biofuel is harvested near the peak of the fat-energy yield of Phase I. The date at which the yield peaks will depend on the specific surface area-weighted mass of carcasses or carcass elements at Day 0; the larger the mass, the later the peak and vice versa. In embodiments, the FB comprises MB, and partial composting comprises composting for less than about 90 days. FIGS. 9 and 10 are bar graphs of ultimate analyses for the SC, CM, and CMH on Day 0; FIGS. 11 and 12 are bar graphs of ultimate analyses for the SC, CM, and CMH on Day 90; and FIGS. 13 and 14 are bar graphs of ultimate analyses for the SC, CM, and CMH on Day 180. FIGS. 15-19 are bar graphs of hydrogen to carbon, nitrogen to carbon, oxygen to carbon, sulphur to carbon, and phosphorus to carbon ratios respectively for SC, CM, and CMH on Days 0, 90, and 180.

### Example 8

### Comparison of Combustion Related Properties of Cattle Manure Biomass by Standard Fuel Analysis with Near Infrared Spectroscopy (NIRS)

Analysis and assessment of combustion-related properties of feedlot biomass with near infrared spectroscopy (NIRS) was investigated. Four of the samples (#170, 171, 172, and 173) were sourced from a typical open soil-surfaced feedlot in Deaf Smith County, and the other sample (#174) was from a nearby open-lot dairy. Samples were collected, dried, milled and prepared for NIRS testing. Five of the samples were split and shipped to the Texas Agricultural Experiment Station/Texas AgriLife Research at Amarillo/Bushland, where they were relabeled and sent to Hazen Research Inc., Golden, Colo. for combustion related testing. The samples sent to Hazen were further prepared by Hazen staff to conform to their testing regiment. The analysis performed for all samples included: ultimate, proximate, chlorine, phosphorus in ash, and a complete ash elemental analysis (equal weight composite sample of #172 and #174 only). Hazen Research Inc. used ASTM lab standards to conduct the necessary analysis. These standards include ASTM#D2013, D2795, D2361, D3172, D3176, D3173, D3174, D3175, D4239, and SW846.

Samples were collected from the aforementioned locations, placed on ice and either transported or mailed (via 2-day priority mail) to the Grazingland Animal Nutrition (GAN) Laboratory in College Station, Tex. Upon receipt at the GAN Lab, samples were brought to room temperature (˜20° C.), crumbled by hand if needed, placed in a plastic cup and scanned with a Perten DA 7200 diode array spectrometer in the 400-2500 nm range. Subsamples were obtained and subjected to gravimetric procedures for quantification of moisture and ash per the recommended methods of AOAC. The remaining sample material was dried overnight at 60° C. in a forced air oven, ground to a 1 mm particle size, dried again at 60° C. in a forced air oven, then re-scanned in this dry, ground state for comparison to the “as received” state. The samples were randomly divided into a calibration (˜75%) and validation (˜25%) set. Both the “as received” and the processed spectra were paired with their respective moisture and ash values and used to build regression equations for the prediction of manure fuel value. Preliminary predictive equations were develop in SAS software and include both stepwise and principal component regression with derivatization and scatter correction as needed. Values reported for NIRS were accomplished with the described equations.

The proximate, ultimate, and heating value analyses for the five samples are shown in Table 15. These analyses also included chlorine and phosphorus concentrations. A composite sample of ash from samples #172 & #174 (60-day stockpiled beef feedlot and dairy manure stockpiled) was analyzed for mineral elements and metals, and these data are shown in Table 16.

The samples all had relatively low moisture (48%) on an as-received basis and moderate to high ash content (33-77%) dry basis. The heating values appeared to be affected by differences in ash content and “age” of materials. HHV ranged from 1,223 (very low) to 5,492 BTU/lb (relatively high) on an as-received basis, and ranged from 1,266-5,955 BTU/lb on a dry basis. The dry ash free basis (DAF) HHV ranged from 6,172 BTU/lb (which is very low) for the 2 year old stockpiled manure to 9,494 BTU/lb (very high) for a 4 day old feedlot manure sample. The samples averaged 8,095 BTU/lb DAF basis.

Relative to heating value and contributing factors, a brief description of analytical results from each of the 5 samples of feedlot and dairy biomass, together with a partial interpretation of the results follows:

Sample #170—The 2-year old stockpiled feedlot manure (sampled Jan. 29, 2007) was exceedingly low in moisture, volatile solids, nitrogen and carbon, and was exceptionally high in ash (79% dry basis); therefore, its higher heating value (HHV) on as-received basis was very low (1,223 BTU/lb), and only 1,266 BTU/lb d.b.

Sample #171—This sample (from Jan. 29, 2007) represented low moisture, moderate ash and carbon, high volatiles, and moderate HHV (4,907 BTU/lb); this was a much better biofuel feedstock than represented by sample #170 from the same feedyard.

Sample #172—Sampled Apr. 18, 2007 from a specific feedpen and as labeled “60 days”, this was stacked in the pen for that period after collection. The sample contained low moisture, relatively low ash (33% d.b.), considering open feedpen source, high volatiles, and moderate carbon. Accordingly, the as-received HHV was relatively high (5,216 BTU/lb w.b.).

Sample #173—This FB was manure sampled Apr. 18, 2007 following harvest 4-days previously from a specific pen; the FB material was similar to Sample #172, except with slightly higher HHV. P was lower than expected for moderate-ash manure.

Sample #174—This represented 60-day old stockpiled DB from a dairy in the Western Panhandle area. While moisture was low, the ash was very high (77% d.b.), and therefore carbon and HHV were exceedingly low. This material was closest to sample #170 in quality (or lack thereof).

Due to analytical high costs, elemental ash analysis was performed only on a composite sample of ash from samples #172 and #174. It proved to be very high in silicon but not as high in phosphorous or potassium as previous samples taken recently from other FB sources in the same sub region. The other elements appeared to be consistent with prior analysis of similar FB and DB ash.

The tabulated summary of dry matter (DM) and organic matter (OM) obtained from the GAN Laboratory Department of Rangeland Ecology at College Station, Tex. are shown in Table 17. The NIRS results are also shown, together with a comparison between these values and the previously-described analytical results from Hazen, from which DM was computed as 100−moisture,% w.b. and OM was computed as 100−ash, % d.b.

Calculated values of HHV were obtained as follows:

HHV BTU/lb=8,500 BTU/lb DAF×((100%−moisture, % w.b.)×(100%−ash, % d.b.)/10,000)

wherein ash and moisture were obtained from the Hazen results. From the Hazen, GAN and NIRS, the results were compared with the current Hazen Lab results, as shown in Table 18. Results shows consistency among the four date sets.

### Example 9

### Separation of Ash with Micrometric Separator

Studies were performed to test the separation of ash from the feedlot biomass using the Micrometric Separator available from DDS Technologies USA, Inc., Boca Raton, Fla. Samples of feedlot biomass were harvested from 12 fly ash-paved pens, placed in a greenhouse, and dried to 10% moisture. The resulting high ash feedlot biomass (HA-FB) was then pre-ground with a hammer mill having a ⅛^{th} inch screen followed by pulverization with a Vortec Impact Mill® until a median particle size of about 60-70% passing a 100 mesh sieve (<149 micron screen). The HA-FB was then sent to DDS Technologies, USA, Inc., where the HA-FB made three passes through a DDS Technologies, USA, Inc.'s Micrometric Separator. The results of the experiment are shown in Table 19.

As shown in Table 19, the mean % ash d.b. of the HA-FB was reduced from about 54% prior to separation using the Micrometric Separator to about 41% after one pass through the Micrometric Separator, 29% after a second pass through the Micrometric Separator, and about 26% after a third pass through the Micrometric Separator.

### Tables

TABLE 1
Feedlot Manure Higher Heating Values (HHV, BTU/lb) as a Function of Ash
Content and Moisture

| | | Ash Content (% d.b.) | | | | | | | |
|---|---|---|---|---|---|---|---|---|---|
| | | 0 | 10 | 20 | 30 | 40 | 50 | 60 | 70 |
| Moisture Content (% w.b.) | 0 | 8,500 | 7,650 | 6,800 | 5,950 | 5,100 | 4,250 | 3,400 | 2,550 |
| | 10 | 7,650 | 6,885 | 6,120 | 5,355 | 4,590 | 3,825 | 3,060 | 2,295 |
| | 20 | 6,800 | 6,120 | 5,440 | 4,760 | 4,080 | 3,400 | 2,720 | 2,040 |
| | 30 | 5,950 | 5,355 | 4,760 | 4,165 | 3,570 | 2,975 | 2,380 | 1,785 |
| | 40 | 5,100 | 4,590 | 4,080 | 3,570 | 3,060 | 2,550 | 2,040 | 1,530 |
| | 50 | 4,250 | 3,825 | 3,400 | 2,975 | 2,550 | 2,125 | 1,700 | 1,275 |
| | 60 | 3,400 | 3,060 | 2,720 | 2,380 | 2,040 | 1,700 | 1,360 | 1,020 |
| | 70 | 2,550 | 2,295 | 2,040 | 1,785 | 1,530 | 1,275 | 1,020 | 765 |

TABLE 2
Analysis of Coal (90%)/Manure (10%) Blend

| | | |
|---|---|---|
| | As Received (n = 5)* | Dry Basis (n = 5) |
| Parameter | Mean ± SD | Mean ± SD |

L Proximate/Elemental Analysis (SPS)*

| | | |
|---|---|---|
| Moisture, % | 8.40 ± 0.16 | — — |
| Ash, % | 10.84 ± 0.11 | 11.84 ± 0.11 |
| Volatiles, % d.b. | 37.75 ± 0.36 | 41.22 ± 0.47 |
| Fixed carbon, % d.b. | 43.01 ± 0.61 | 46.94 ± 0.56 |
| Sulfur, % | 0.48 ± 0.02 | 0.52 ± 0.02 |
| High Heating Value | 10,164 ± 117 | 11,096 ± 111 |
| (HHV), BTU/lb | | |
| High Heating Value | — — | 12,586 ± 111 |
| (HHV), BTU/lb, DAF | | |

IL Ultimate Analysis (CTE)**

| | | |
|---|---|---|
| Moisture, % | 7.86 ± 0.27 | — — |
| Ash, % | 11.35 ± 0.19 | 12.32 ± 0.22 |
| Carbon, % | 60.10 ± 0.62 | 65.22 ± 0.57 |
| Hydrogen, % | 4.31 ± 0.02 | 4.67 ± 0.02 |
| Nitrogen, % | 1.06 ± 0.01 | 1.15 ± 0.01 |
| Sulfur, % | 0.50 ± 0.01 | 0.54 ± 0.01 |
| Oxygen (diff.), % | 14.84 ± 0.40 | 16.11 ± 0.45 |
| Totals | 100.02 — | 100.01 — |

*Data are mean ± standard deviation of constituents from one composite sample from 5 of the 10 fuel blend test drums (#114, 117, 119, 121, and 123) prepared by USDOE/NETL for cofiring combustion test in their DOE Combustion and Environmental Research Facility (CERF).

TABLE 3
Constituent Values of Beef Cattle Manure Collected From
Feedyard Pen Surface Treatments

| | | Control Pens | | | Crushed Ash Pens | | |
|---|---|---|---|---|---|---|---|
| Constituent | Units | Mean | S.D. | n | Mean | S.D. | n |
| WET BASIS | | | | | | | |
| Moisture | % | 23.10 a | 10.7 | 27 | 27.98 a | 8.96 | 27 |
| Ash | % | 51.00 a | 12.37 | 27 | 29.70 b | 9.99 | 27 |
| Volatile Solids | % | 26.77 a | 7.52 | 17 | 42.50 b | 7.91 | 19 |
| Carbon | % | 13.65 a | 3.21 | 4 | 21.15 b | 3.48 | 4 |
| Heating Value | BTU/lb | 1,982 a | 598 | 14 | 3,520 b | 661 | 12 |
| DRY BASIS | | | | | | | |
| Ash | % | 65.71 a | 9.85 | 27 | 39.25 b | 11.05 | 27 |
| Volatile Solids | % | 34.97 a | 10.01 | 17 | 59.10 b | 10.69 | 19 |
| Nitrogen | % | 1.51 a | 0.57 | 30 | 2.83 b | 0.63 | 34 |
| Phosphorus | % | 0.477 a | 0.178 | 13 | 0.967 b | 0.175 | 15 |
| Potassium | % | 1.63 a | 0.48 | 13 | 2.75 b | 0.51 | 15 |
| Carbon | % | 15.78 a | 4.29 | 4 | 26.21 b | 5.76 | 4 |
| Calcium | % | 2.91 a | 0.9 | 13 | 2.42 a | 0.55 | 15 |
| Magnesium | % | 0.337 a | 0.098 | 13 | 0.617 b | 0.088 | 15 |
| Sodium | ppm | 5,482 a | 1,944 | 13 | 10,380 b | 1,805 | 15 |
| Zinc | ppm | 68.47 a | 22.95 | 13 | 126.91 b | 23.86 | 15 |
| Iron | ppm | 2,256 a | 1,369 | 13 | 968 b | 536 | 15 |
| Copper | ppm | 14.14 a | 6.19 | 13 | 28.63 b | 6.36 | 15 |
| Manganese | ppm | 117.4 a | 25.55 | 13 | 107.73 a | 14.75 | 15 |
| Heating Value | BTU/lb | 2,601 a | 991 | 14 | 4,842 b | 1,222 | 12 |
| DAF Heating Value | BTU/lb | 7,835 a | 1,018 | 14 | 8,656 b | 610 | 12 |

a and b constituent means followed by the same letter are not statistically different.

TABLE 4
Nutrient Analysis of Cattle Feedlot Manure

| | Pen Manure | Composted Manure | Stockpiled Manure |
|---|---|---|---|
| | 14 Feedlots | 3 Feedlots | 4 Feedlots |
| Parameter | (123 Samples) | (26 Samples) | (32 Samples) |
| Moisture, % w.b. | 20.6 ± 7.8 | 17.2 ± 8.9 | 20.0 ± 9.8 |
| Nitrogen, N, % d.b. | 2.28 ± 0.59 | 1.94 ± 0.43 | 1.39 ± 0.49 |
| Phosphorus, P, % d.b. | 0.76 ± 0.12 | 0.90 ± 0.12 | 0.68 ± 0.26 |
| Potassium, K, % d.b. | 2.29 ± 0.32 | 2.53 ± 0.38 | 1.43 ± 0.53 |
| Calcium, Ca, % d.b. | 3.43 ± 0.90 | 4.15 ± 0.64 | 4.21 ± 1.80 |
| Magnesium, Mg, % d.b. | 0.78 ± 0.21 | 0.94 ± 0.14 | 0.73 ± 0.31 |
| Sodium, Na, ppm d.b. | 10,152 ± 5,084 | 10,463 ± 4,476 | 6,637 ± 4,812 |
| Zinc, Zn, ppm d.b. | 254 ± 75 | 200 ± 14 | 132 ± 44 |
| Iron, Fe, ppm d.b. | 1,433 ± 405 | 2,532 ± 1,596 | 2,306 ± 2,528 |
| Copper, Cu, ppm d.b. | 44 ± 13 | 43 ± 7 | 30 ± 12 |
| Manganese, Mn, ppm d.b. | 191 ± 52 | 269 ± 49 | 246 ± 87 |

TABLE 5
Fly Ash Analysis

| PARAMETER | RESULTS | ASTM C618 SPE |
|---|---|---|
| CHEMICAL ANALYSIS | | |
| ALUMINUM OXIDE | 20.14 | |
| IRON OXIDE | 5.41 | |
| SILICA | 36.39 | |
| SUM OF AL, FE, SI OXIDES | 61.94 | 50 MIN |
| CALCIUM OXIDE | 26.75 | |
| MAGNESIUM OXIDE | 5.33 | |
| SULFUR TRIOXIDE | 1.99 | 5.0 MAX |
| AVAILABLE ALKALI | 1.3 | 1.5 MAX |
| AVAILABLE SODIUM OXIDE | 1.1 | |
| AVAILABLE POTASSIUM OXIDE | 0.25 | |
| PHYSICAL ANALYSIS | | |
| % RETAINED ON 325 SIEVE | 19.85 | 34.0 MAX |
| % MOISTURE IN ASH | 0.04 | 3.0 MAX |
| % LOST ON IGNITION | 0.27 | 6.0 MAX |
| H2O REQUIREMENT-% OF CONTROL | 100.4 | 10.5 MAX |
| AUTOCLAVE EXPANSION | 0.048 | 0.8 MAX |
| SPECIFIC GRAVITY | 2.61 | |
| POZZOLANIC ACTIVITY INDEX | | |
| % OF CEMENT CONTROL | 99.34 | 75 MIN |
| PSI OF CEMENT CONTROL | 3763 | |
| PSI OF SAMPLE | 3738 | |

TABLE 6
Proximate and Ultimate Analysis of As-Collected (Un-composted) Feedlot Biomass
Harvested from (a) Soil-Surfaced (SS) Cattle Feedpens (n = 6) (HA-FB) and (b) Fly Ash (FA)
Feedpens (n = 12) (LA-FB)

| | Soil-Surfaced Feedpens (n = 6), HA-FB | | | | Fly Ash-Surfaced Feedpens (n = 12), LA-FB | | | |
|---|---|---|---|---|---|---|---|---|
| | Harvesting Date = Jun. 10, 2005 | | | | Harvesting Date = Jun. 1, 2005 | | | |
| | SS 101-103 | | SS101-103 | | FA104-106 | | FA104-106 | |
| | As-Received % | | Dry, % | | As-Received % | | Dry, % | |
| Parameter | Mean | Std. Dev. | Mean | Std. Dev. | Mean | Std. Dev. | Mean | Std. Dev. |
| Proximate: | | | | | | | | |
| Moisture | 19.81 | 1.24 | 0 | 0 | 20.27 | 1.27 | 0 | 0 |
| Ash | 47.10 | 1.29 | 58.73 | 1.65 | 16.10 | 0.73 | 20.20 | 1.11 |
| Volatile | 27.08 | 1.25 | 33.77 | 1.26 | 51.47 | 1.34 | 64.56 | 0.94 |
| Fixed C | 6.02 | 0.36 | 7.50 | 0.45 | 12.16 | 0.40 | 15.24 | 0.27 |
| Total | 100.01 | | 100.00 | | 100.00 | | 100.00 | |
| Heating Value | | | | | | | | |
| HHV, BTU/lb | 2710 | 34 | 3380 | 14 | 5764 | 147 | 7229 | 92 |
| MMF, BTU/lb | 5505 | 174 | 9259 | 457 | 6969 | 133 | 9247 | 26 |
| MAF/DAF, | | | 8200 | 327 | | | 9059 | 13 |
| BTU/lb | | | | | | | | |
| Ultimate: | | | | | | | | |
| Moisture | 19.81 | 1.24 | 0 | 0 | 20.27 | 1.23 | 0 | 0 |
| Carbon | 17.39 | 0.9 | 21.69 | 1.14 | 34.35 | 0.77 | 43.09 | 0.49 |
| Hydrogen | 2.1 | 0.10 | 2.62 | 0.13 | 4.17 | 0.11 | 5.22 | 0.05 |
| Nitrogen | 1.56 | 0.04 | 1.94 | 0.07 | 2.48 | 0.04 | 3.11 | 0.03 |
| Sulfur | 0.34 | 0.02 | 0.42 | 0.02 | 0.53 | 0.02 | 0.67 | 0.01 |
| Ash | 47.1 | 1.29 | 58.73 | 1.65 | 16.10 | 0.73 | 20.20 | 1.11 |
| Oxygen (diff.) | 11.7 | 0.82 | 14.59 | 0.81 | 22.10 | 0.80 | 27.70 | 0.83 |
| Total | 100.00 | | 99.99 | | 100.00 | | 99.99 | |
| Chlorine | SS 101-103 Composite | | | | FA 104-106 Composite | | | |
| Chlorine, Cl | 0.301 | | 0.375 | | 0.302 | | 0.377 | |

| | | | | |
|---|---|---|---|---|
| Phosphorus | | | | |
| Phosphorus (Ash Basis), P205, % | 2.74 | 0.08 | 12.87 | 0.85 |
| Phosphorus (Dry Basis), P205, % | 1.61 | 0.04 | 2.59 | 0.04 |
| Contaminants, Energy Basis: | | | | |
| Ash, lbs/MM BTU | 173.78 | 5.13 | 27.96 | 1.89 |
| SO2, lbs/MM BTU | 2.51 | 0.13 | 1.86 | 0.05 |

TABLE 7
Elemental Analysis of FB Sample Ash from As-Collected (Un-Composted) FB from
Un-Paved and Paved Pens (HA-FB and LA-FB), from Partially Composted
FB (HA-FB-PC and LA-FB-PC), and from Texas Lignite (TXL) and PRB Coal

| | | | | | |
|---|---|---|---|---|---|
| HA-FB, | LA-FB, | HA-FB-PC, | LA-FB-PC, | TXL | PBB Coal |
| %, Dry | %, Dry | %, Dry | %, Dry | %, Dry | %, Dry |
| Basis | Basis | Basis | Basis | Basis | Basis |

Ash Elemental Analysis* (%), Equal-Weight-Composite (n = 1)

| | | | | | | |
|---|---|---|---|---|---|---|
| Silicon, Si02 | 64.68 | 25.55 | 65.55 | 20.78 | 48.72 | 31.73 |
| Aluminum, | 7.72 | 1.94 | 11.2 | 4.94 | 16.04 | 17.27 |
| Al203 | | | | | | |
| Titanium, | 0.44 | 0.27 | 0.52 | 0.22 | 0.85 | 1.35 |
| Ti02 | | | | | | |
| Iron, Fe203 | 2.90 | 1.37 | 2.99 | 1.71 | 7.44 | 4.61 |
| Calcium, Ca0 | 7.09 | 20.20 | 7.47 | 21.0 | 11.70 | 22.20 |
| Magnesium, | 2.34 | 7.17 | 2.29 | 7.54 | 1.93 | 5.62 |
| Mg0 | | | | | | |
| Sodium, | 1.38 | 4.94 | 1.38 | 5.26 | 0.29 | 1.43 |
| Na20 | | | | | | |
| Potassium, | 4.50 | 12.70 | 4.66 | 14.60 | 0.61 | 0.67 |
| K20 | | | | | | |
| Phosphorus, | 2.81 | 11.11 | 2.43 | 13.77 | 0.10 | 0.80 |
| P205 | | | | | | |
| Sulfur, S03 | 1.08 | 4.46 | 1.30 | 4.47 | 10.80 | 10.40 |
| Chlorine, Cl | 0.68 | 5.02 | 0.41 | 5.07 | <0.01 | <0.01 |
| Carbon | 1.35 | 1.71 | 0.51 | 0.59 | 0.08 | 0.37 |
| Dioxide, C02 | | | | | | |
| Total Ash | 96.95 | 96.44 | 100.71 | 99.95 | 98.56 | 96.45 |
| Analysis | | | | | | |

Metals in Ash (mg/kg) equal-weight (n = 1)

| | | | | | | |
|---|---|---|---|---|---|---|
| Arsenic | 4.12 | 3.95 | 3.85 | 2.81 | 24.7 | 17.6 |
| Barium | 669 | 2,620 | 800 | 700 | 1,590 | 6,230 |
| Cadmium | <1 | 2 | 3.8 | 8.2 | 3.4 | 5.2 |
| Chromium | <20 | 20 | 30 | 40 | 98 | 110 |
| Lead | 20 | 20 | 27 | 15 | 47 | 130 |
| Mercury | <0.01 | <0.01 | 0.03 | 0.04 | 0.01 | <0.01 |
| Selenium | <2 | 2 | <2 | 4 | <2 | <2 |
| Silver | <2 | <2 | <2 | <2 | <2 | <2 |
| Total Metals | 693.12 | 2,657.96 | 864.68 | 770.05 | 1,763.11 | 6,492.80 |
| in Ash | | | | | | |

TABLE 8
Comparison of Proximate and Elemental Analyses of Composted Feedlot Manure
after 1, 32, and 125 Days of Composting

| | |
|---|---|
| Manure Only | Manure + <5% v/v Crop Res. |

| | | | | | | |
|---|---|---|---|---|---|---|
| | Sampling | No. Days | As Received | Dry Basis | As Received | Dry Basis |
| Parameter | Date | Composting | Mean (SD) | Mean (SD) | Mean (SD) | Mean (SD) |

I. Proximate/Elemental Analysis (SPS)*

| | | | | | | |
|---|---|---|---|---|---|---|
| Proximate Analysis | | | | | | |
| Moisture, % | Dec. 5, 1998 | 1 | 40.18 (0.98) | 0.0 (0) | 35.81 (0.17) | 0.0 (0) |
| | Jan. 5, 1999 | 32 | 32.5 (3.71) | 0.0 (0) | 31.73 (2.47) | 0.0 (0) |
| | Apr. 8, 1999 | 125 | 32.43 (0.29) | 0.0 (0) | 28.49 (1.75) | 0.0 (0) |
| Ash, % | Dec. 5, 1998 | 1 | 21.47 (0.40) | [symbol] 5.90 (0.10) | 25.63 (0.06) | 39.97 (0.06) |
| | Jan. 5, 1999 | 32 | 30.3 (2.11) | 44.6 (1.2) | 30.57 (2.59) | 44.7 (2.3) |
| | Apr. 8, 1999 | 125 | 32.9 (0.98) | 48.7 (1.64) | 38.9 (2.41) | 54.37 (2.33) |
| Volatiles, % d.b. | Dec. 5, 1998 | 1 | | 50.22 (0.94) | | 47.61 (1.09) |
| | Jan. 5, 1999 | 32 | | 42.3 (1.27) | | 42.77 (0.51) |
| | Apr. 8, 1999 | 125 | | 39.14 (0.61) | | 37.89 (0.52) |
| Fixed carbon, % d.b. | Dec. 5, 1998 | 1 | | 11.27 (0.26) | | 10.36 (0.23) |
| | Jan. 5, 1999 | 32 | | 10.08 (0.17) | | 9.43 (0.19) |
| | Apr. 8, 1999 | 125 | | 9.45 (0.43) | | 9.70 (0.58) |
| Sulfur, % | Dec. 5, 1998 | 1 | 0.45 (0.02) | 0.76 (0.03) | 0.47 (0.02) | 0.73 (0.04) |
| | Jan. 5, 1999 | 32 | 0.51 (0.03) | 0.75 (0.03) | 0.49 (0.06) | 0.71 (0.02) |
| | Apr. 8, 1999 | 125 | 0.51 (0.01) | 0.75 (0.01) | 0.49 (0.02) | 0.68 (0.01) |
| HHV, BTU/lb | Dec. 5, 1998 | 1 | 3,445 (85) | 5,762 (220) | 3,277 (51) | 5,104 (73) |
| | Jan. 5, 1999 | 32 | 3,159 (164) | 4,656 (92) | 3,204 (161) | 4,702 (390) |
| | Apr. 8, 1999 | 125 | 2,944 (168) | 4,308 (156) | 3,051 (13) | 4,268 (117) |
| Ash Analysis | | | | | | |
| Sodium, % of Ash | Dec. 5, 1998 | 1 | | 2.93 (0.12) | | 3.30 (0.11) |
| | Jan. 5, 1999 | 32 | | 2.18 (0.03) | | 2.55 (0.17) |
| | Apr. 8, 1999 | 125 | | 2.44 (0.07) | | 2.20 (0.11) |
| Magnesium, % of Ash | Dec. 5, 1998 | 1 | | 5.08 (0.02) | | 4.34 (0.15) |
| | Jan. 5, 1999 | 32 | | 4.40 (0.36) | | 4.53 (0.09) |
| | Apr. 8, 1999 | 125 | | 4.53 (0.19) | | 4.00 (0.13) |
| Potassium, % of Ash | Dec. 5, 1998 | 1 | | 11.71 (0.22) | | 10.65 (0.33) |
| | Jan. 5, 1999 | 32 | | 8.95 (0.27) | | 8.90 (0.48) |
| | Apr. 8, 1999 | 125 | | 8.66 (0.03) | | 8.18 (0.36) |
| Calcium, % of Ash | Dec. 5, 1998 | 1 | | 13.62 (0.41) | | 11.73 (0.03) |
| | Jan. 5, 1999 | 32 | | 12.25 (0.48) | | 11.64 (0.43) |
| | Apr. 8, 1999 | 125 | | 12.82 (0.42) | | 13.22 (0.77) |

*Data are results from three subsamples composited from ~20 probe subsamples from each manure windrow. [symbol]
indicates data missing or illegible when filed

TABLE 9
Comparison of Ultimate Analysis of Composted Feedlot Manure after 1, 32, and
125 Days of Windrow Composting

| | | | | | Manure + | |
|---|---|---|---|---|---|---|
| | Sampling | No. Days | Manure only | | <5% v/v Crop Res. | |
| Parameter | Date | Composting | As Received | Dry Basis | As Received | Dry Basis |

II. Ultimate Analysis (CTE)**

| | | | | | | |
|---|---|---|---|---|---|---|
| Moisture, % | Dec. 5, 1998 | 1 | 38.61 | 0 | 36.15 | 0 |
| | Jan. 5, 1999 | 32 | 31.96 | 0 | 30.72 | 0 |
| | Apr. 8, 1999 | 125 | 31.18 | 0 | 27.32 | 0 |
| Ash, % | Dec. 5, 1998 | 1 | 24.79 | 40.38 | 27.3 | 42.76 |
| | Jan. 5, 1999 | 32 | 30.71 | 45.14 | 28.54 | 41.2 |
| | Apr. 8, 1999 | 125 | 33.36 | 48.47 | 38.14 | 52.48 |
| Carbon, % | Dec. 5, 1998 | 1 | 18.17 | 29.59 | 18.89 | 29.58 |
| | Jan. 5, 1999 | 32 | 19.76 | 29.04 | 19.96 | 28.81 |
| | Apr. 8, 1999 | 125 | 16.81 | 24.42 | 17.58 | 24.2 |
| Hydrogen, % | Dec. 5, 1998 | 1 | 2.06 | 3.35 | 2.19 | 3.43 |
| | Jan. 5, 1999 | 32 | 2.2 | 3.23 | 2.16 | 3.12 |
| | Apr. 8, 1999 | 125 | 1.65 | 2.4 | 1.82 | 2.5 |
| Nitrogen, % | Dec. 5, 1998 | 1 | 1.57 | 2.55 | 1.48 | 2.32 |
| | Jan. 5, 1999 | 32 | 1.67 | 2.46 | 1.62 | 2.34 |
| | Apr. 8, 1999 | 125 | 1.61 | 2.34 | 1.69 | 2.33 |
| Sulfur, % | Dec. 5, 1998 | 1 | 0.5 | 0.81 | 0.51 | 0.8 |
| | Jan. 5, 1999 | 32 | 0.56 | 0.82 | 0.53 | 0.76 |
| | Apr. 8, 1999 | 125 | 0.6 | 0.87 | 0.6 | 0.83 |
| Oxygen, % (diff.) | Dec. 5, 1998 | 1 | 14.3 | 23.32 | 13.48 | 21.11 |
| | Jan. 5, 1999 | 32 | 13.14 | 19.31 | 16.47 | 23.77 |
| | Apr. 8, 1999 | 125 | 14.79 | 21.49 | 12.85 | 17.68 |

**Data are CTE analysis of one composite of the three subsamples analyzed by SPS for proximate analysis.

TABLE 10
Manure Analysis Summary for Uncomposted, Partially Composted (PC), and Finished
Composted (FC) Biomass Following 204, 297, and 328 Days of Bin-Storage Under Roof

| | | | | | Partially Composted | |
|---|---|---|---|---|---|---|
| | | | | | Manure (PC) + | |
| | Uncomposted Manure (RM) + | | | | (297 days | |
| | (328 days In-Bin Storage) | | | | In-Bin Storage) | |
| | Manure Only | | <5% Crop Residues | | Manure Only | |
| Parameter | As Received | Dry Basis | As Received | Dry Basis | As Received | Dry Basis |

I. Proximate/Elemental Analysis (SPS)*

| | | | | | | |
|---|---|---|---|---|---|---|
| Moisture, % | 23.27 | — | 10.90 | — | 9.25 | — |
| Ash, % | 32.30 | 42.10 | 39.60 | 44.50 | 44.60 | 49.20 |
| Volatiles, % d.b. | — | 46.47 | — | 44.96 | — | 41.34 |
| Fixed carbon, % d.b. | — | 11.43 | — | 10.54 | — | 9.46 |
| Sulfur, % | 0.61 | 0.79 | 0.54 | 0.61 | 0.64 | 0.70 |
| HHV, BTU/lb | 3,886 | 5,064 | 4,115 | 4,618 | 3,743 | 4,125 |
| HHV, BTU/lb, DAF | — | 8,746 | — | 8,321 | — | 8,120 |

II. Ultimate Analysis (CTE)*

| Moisture, % | 22.97 | — | 10.83 | — | 9.12 | — |
|---|---|---|---|---|---|---|
| Ash, % | 32.95 | 42.78 | 39.81 | 44.65 | 44.99 | 49.50 |
| Carbon, Fixed % | 23.73 | 30.80 | 26.41 | 29.62 | 23.87 | 26.26 |
| Hydrogen, % | 2.60 | 3.37 | 3.04 | 3.41 | 2.58 | 2.84 |
| Nitrogen, % | 2.21 | 2.87 | 1.98 | 2.22 | 2.03 | 2.23 |
| Sulfur, % | 0.72 | 0.94 | 0.73 | 0.83 | 0.73 | 0.80 |
| Oxygen (diff.), % | 14.82 | 19.24 | 17.20 | 19.28 | 16.68 | 18.37 |
| Totals | 100.00 | 100.00 | 100.00 | 100.01 | 100.00 | 100.00 |
| | Partially Composted | | | | | |
| | Manure (PC) + | | | | | |
| | (297 days | | Finished Compost (FC) + | | | |
| | In-Bin Storage) | | (204 days In-Bin Storage) | | | |
| | <5% Crop Residues | | Manure Only | | <5% Crop Residues | |
| Parameter | As Received | Dry Basis | As Received | Dry Basis | As Received | Dry Basis |

I. Proximate/Elemental Analysis (SPS)*

| | | | | | | |
|---|---|---|---|---|---|---|
| Moisture, % | 18.09 | — | 20.85 | — | 20.52 | — |
| Ash, % | 40.10 | 49.00 | 40.20 | 50.80 | 41.20 | 51.80 |
| Volatiles, % d.b. | — | 41.26 | — | 39.86 | — | 38.84 |
| Fixed carbon, % d.b. | — | 9.74 | — | 9.34 | — | 9.36 |
| Sulfur, % | 0.57 | 0.70 | 0.68 | 0.86 | 0.64 | 0.80 |
| HHV, BTU/lb | 3,465 | 4,230 | 3,277 | 4,140 | 2,985 | 3,756 |
| HHV, BTU/lb, DAF | — | 8,294 | — | 8,415 | — | 7,793 |

II. Ultimate Analysis (CTE)*

| | | | | | | |
|---|---|---|---|---|---|---|
| Moisture, % | 17.90 | — | 20.94 | — | 20.14 | — |
| Ash, % | 40.08 | 48.82 | 39.40 | 49.83 | 42.53 | 53.25 |
| Carbon, Fixed % | 21.90 | 26.67 | 21.85 | 27.64 | 19.62 | 24.57 |
| Hydrogen, % | 2.41 | 2.93 | 2.35 | 2.97 | 2.03 | 2.54 |
| Nitrogen, % | 1.86 | 2.26 | 2.00 | 2.53 | 1.81 | 2.27 |
| Sulfur, % | 0.65 | 0.79 | 0.68 | 0.86 | 0.63 | 0.79 |
| Oxygen (diff.), % | 15.20 | 18.53 | 12.78 | 16.17 | 13.24 | 16.58 |
| Totals | 100.00 | 100.00 | 100.00 | 100.00 | 100.00 | 100.00 |

*Data are results from one composite sample from ~20 probe subsamples per treatment.

TABLE 11
Proximate and Ultimate Analyses of Partially-Composted (PC) FB Harvested
from Soil-Surfaced (SS) Feedpens (n = 6) HA-FB vs. PC FB Harvested from Fly Ash (FA)-
Surfaced Feedpens (n = 12) LA-FB

| | Soil-Surfaced Feedpens (n = 6) | | | | Fly Ash Surfaced Feedpens (n = 12) | | | |
|---|---|---|---|---|---|---|---|---|
| | HA-FB-PC, 51 Days Composting | | | | LA-FB-PC, 55 Days Composting | | | |
| | SS 107-109 | | SS 107-109 | | FA 110-112 | | FA 110-112 | |
| | As-Received % | | Dry, % | | As-Received % | | Dry, % | |
| Parameter | Mean | Std. Dev. | Mean | Std. Dev. | Mean | Std. Dev. | Mean | Std. Dev. |
| Proximate: | | | | | | | | |
| Moisture | 17.00 | 0.26 | 0 | 0 | 19.64 | 2.54 | 0 | 0 |
| Ash | 53.85 | 0.77 | 64.88 | 0.74 | 16.50 | 0.28 | 20.53 | 0.52 |
| Volatile | 25.79 | 1.04 | 31.07 | 1.31 | 52.33 | 2.12 | 65.11 | 0.59 |
| Fixed C | 3.36 | 0.78 | 4.05 | 0.95 | 11.54 | 0.32 | 14.36 | 0.28 |
| Total | 100.00 | | 100.00 | | 100.01 | | 100.00 | |
| Heating Value: | | | | | | | | |
| HHV, BTU/lb | 2239 | 49 | 2697 | 60 | 5704 | 192 | 7097 | 17 |
| MMF, BTU/lb | 5336 | 134 | 9015 | 228 | 6933 | 250 | 9119 | 45 |
| MAF/DAF, | | | 7682 | 169 | | | 8931 | 38 |
| BTU/lb | | | | | | | | |
| Ultimate: | | | | | | | | |
| Moisture | 17.00 | 0.26 | 0 | 0 | 19.64 | 2.54 | 0 | 0 |
| Carbon | 14.92 | 0.16 | 17.97 | 0.25 | 33.79 | 1.10 | 42.05 | 0.14 |
| Hydrogen | 1.39 | 0.08 | 1.68 | 0.10 | 3.65 | 0.30 | 4.55 | 0.29 |
| Nitrogen | 1.13 | 0.02 | 1.36 | 0.03 | 1.97 | 0.07 | 2.45 | 0.02 |
| Sulfur | 0.31 | 0.02 | 0.38 | 0.02 | 0.51 | 0.02 | 0.64 | 0.04 |
| Ash | 53.85 | 0.77 | 64.88 | 0.74 | 16.50 | 0.28 | 20.53 | 0.52 |
| Oxygen (diff.) | 11.40 | 0.27 | 13.73 | 0.37 | 23.94 | 1.03 | 29.78 | 0.36 |
| Total | 100.00 | | 100.00 | | 100.00 | | 100.00 | |

| | | |
|---|---|---|
| Chlorine | SS 107-109 Composite | FA 110-112 Composite |

| | | | | |
|---|---|---|---|---|
| Chlorine, CI | 0.281 | 0.338 | 0.727 | 0.905 |

| | | | | |
|---|---|---|---|---|
| Phosphorus | | | | |
| Phosphorus (Ash Basis), P205, % | 2.43 | 0.05 | 13.30 | 0.69 |
| Phosphorus (Dry Basis), P205, % | 1.57 | 0.01 | 2.73 | 0.11 |
| Contaminants, Energy Basis: | | | | |
| Ash, lbs/MM BTU | 240.56 | 7.13 | 28.94 | 0.81 |
| SO2, lbs/MM BTU | 2.79 | 0.13 | 1.79 | 0.11 |

TABLE 12
Comparison (Dry Basis) of Un-Composted and Partially-Composted FB from Soil-
Surfaced and Fly Ash-Surfaced Feedpens

| | Soil-Surfaced | | | | | | | |
|---|---|---|---|---|---|---|---|---|
| | (SS) Feedpens (n = 6) HA-FB | | | | Fly Ash-Surfaced (FA) | | | |
| | | | Aug. 2, 2005 - | | | | Aug. 2,2005 - | |
| | Before composting | | 51 day compost | | Before composting | | 55 day compost | |
| | SS 101-103 | | SS 107-109 | | FA 104-108 | | FA 110-112 | |
| | Dry, % | | Dry, % | | Dry, % | | Dry, % | |
| Parameter | Mean | Std. Dev. | Mean | Std. Dev. | Mean | Std. Dev. | Mean | Std. Dev. |
| Proximate: | | | | | | | | |
| Moisture | 0.00 | 0.00 | 0.00 | 0.00 | 0.00 | 0.00 | 0.00 | 0.00 |
| Ash | 58.73 | 1.65 | 64.88 | 0.74 | 20.20 | 1.11 | 20.53 | 0.52 |
| Volatile | 33.77 | 1.26 | 31.07 | 1.31 | 64.56 | 0.94 | 65.11 | 0.59 |
| Fixed C | 7.50 | 0.45 | 4.05 | 0.95 | 15.24 | 0.27 | 14.36 | 0.28 |
| Total | 100.00 | | 100.00 | | 100.00 | | 100.00 | |
| HHV, BTU/lb | 3.380 | 14 | 2697 | 60 | 7229 | 92 | 7097 | 17 |
| MMF, BTU/lb | 9.259 | 457 | 9015 | 228 | 9247 | 26 | 9119 | 45 |
| MAF/DAF, | 8.200 | 327 | 7682 | 169 | 9056 | 13 | 8931 | 38 |
| BTU/lb | | | | | | | | |
| Ultimate: | | | | | | | | |
| Moisture | 0.00 | 0.00 | 0.00 | 0.00 | 0.00 | 0.00 | 0.00 | 0.00 |
| Carbon | 21.69 | 1.14 | 17.97 | 0.25 | 43.09 | 0.49 | 42.05 | 0.14 |
| Hydrogen | 2.62 | 0.13 | 1.68 | 0.10 | 5.22 | 0.05 | 4.55 | 0.29 |
| Nitrogen | 1.94 | 0.07 | 1.36 | 0.03 | 3.11 | 0.03 | 2.45 | 0.02 |
| Sulfur | 0.42 | 0.02 | 0.38 | 0.02 | 0.67 | 0.01 | 0.64 | 0.04 |
| Ash | 58.73 | 1.65 | 64.88 | 0.74 | 20.20 | 1.11 | 20.53 | 0.52 |
| Oxygen (diff.) | 14.59 | 0.81 | 13.73 | 0.37 | 27.70 | 0.63 | 29.78 | 0.36 |
| Total | 99.99 | | 100.00 | | 99.89 | | 100.00 | |
| Chlorine One | | | | | | | | |
| Composite | | | | | | | | |
| of 3 samples | | | | | | | | |
| per FB Type | | | | | | | | |
| Chlorine, CI | 0.375 | | 0.338 | | 0.377 | | 0.905 | |
| Phosphorus, | | | | | | | | |
| P₂o5% | | | | | | | | |
| P-Ash Basis | 2.74 | 0.08 | 2.43 | 0.05 | 12.87 | 0.85 | 13.30 | 0.69 |
| P-Dry Basis | 1.04 | 0.04 | 1.57 | 0.01 | 2.59 | 0.04 | 2.73 | 0.11 |
| Contaminants, | | | | | | | | |
| Energy Basis: | | | | | | | | |
| Ash, lbs/MM | 173.78 | 5.13 | 240.66 | 7.13 | 27.96 | 1.89 | 28.94 | 0.81 |
| BTU | | | | | | | | |
| SO2, lbs/MM | 2.51 | 0.13 | 2.79 | 0.13 | 1.86 | 0.05 | 1.79 | 0.11 |
| BTU | | | | | | | | |

TABLE 13
Texas Lignite (TXL) and Wyoming Powder River Basin (PRB) Coal

| | TXL | | TXL | | PRB | | | |
|---|---|---|---|---|---|---|---|---|
| | 113-115 (n = 3) | | 113-115 (n = 3) | | 116-118 (n = 3) | | PRB 116-118 (n = 3) | |
| | As-Received % | | Dry, % | | As-Received % | | Dry, % | |
| Parameter | Mean | Std. Dev. | Mean | Std. Dev. | Mean | Std. Dev. | Mean | Std. Dev. |
| Proximate: | | | | | | | | |
| Moisture | 38.34 | 0.34 | 0.00 | 0.00 | 32.88 | 0.36 | 0.00 | 0.00 |
| Ash | 11.46 | 0.50 | 18.59 | 0.85 | 5.64 | 2.11 | 8.40 | 3.11 |
| Volatile | 24.79 | 0.26 | 40.20 | 0.53 | 28.49 | 0.62 | 42.45 | 1.02 |
| 0.45Fixed C | 25.41 | 0.63 | 41.21 | 0.80 | 32.99 | 1.31 | 49.15 | 2.15 |
| Total | 100.00 | | 100.00 | | 100.00 | | 100.00 | |
| Heating Value | | | | | | | | |
| HHV, BTU/lb | 6143 | 127 | 9962 | 170 | 7823 | 282 | 11657 | 455 |
| MMF, BTU/lb | 7003 | 109 | 12487 | 70 | 8328 | 121 | 12828 | 81 |
| MAF/DAF, | | | 12236 | 84 | | | 12724 | 97 |
| BTU/ lb | | | | | | | | |
| Ultimate: | | | | | | | | |
| Moisture | 38.34 | 0.34 | 0.00 | 0.00 | 32.88 | 0.36 | 0.00 | 0.00 |
| Carbon | 37.18 | 0.66 | 60.30 | 0.92 | 46.52 | 1.74 | 69.32 | 2.82 |
| Hydrogen | 2.12 | 0.08 | 3.44 | 0.14 | 2.73 | 0.07 | 4.06 | 0.13 |
| Nitrogen | 0.68 | 0.01 | 1.11 | 0.02 | 0.66 | 0.03 | 0.98 | 0.04 |
| Sulfur | 0.61 | 0.09 | 0.98 | 0.15 | 0.27 | 0.02 | 0.41 | 0.03 |
| Ash | 11.46 | 0.50 | 18.59 | 0.85 | 5.65 | 2.11 | 8.40 | 3.11 |
| Oxygen (diff.) | 9.61 | 0.32 | 15.58 | 0.44 | 11.29 | 0.14 | 16.83 | 0.29 |
| Total | 100.00 | | 100.00 | | 100.00 | | 100.00 | |
| Chlorine One | | | | | | | | |
| Composite | | | | | | | | |
| of 3 samples | | | | | | | | |
| Chlorine, CI | 0.01 | | 0.016 | | 0.009 | | 0.013 | |

| | | | | |
|---|---|---|---|---|
| Phosphorus | | | | |
| P-Ash Basis, P₂05, % | 0.13 | 0.01 | 0.57 | 0.14 |
| P-Dry Basis, P₂05, % | 0.02 | 0.00 | 0.05 | 0.01 |
| Contaminants, Energy Basis: | | | | |
| Ash, lbs/MM | 18.67 | 1.17 | 7.28 | 3.02 |
| BTU | | | | |
| SO2, lbs/MM | 1.98 | 0.32 | 0.70 | 0.02 |
| BTU | | | | |

TABLE 14
Partial Results of Baseline NOx Emissions from Cattle Feedlot (PC) Manure
Reburn Experiments-150 kW Pilot Plant, May 20-23, 2002

| | | Natural Gas | | | | Measured NOx | | |
|---|---|---|---|---|---|---|---|---|
| | | Firing Rate, | Primary Burner | | | w/Manure Reburn | | |
| Date | Time | BTU/hr | PPM NOx | lbs NOx/MMBTU | Ratio* | PPM NOx | lbs NOx/MMBTU | Ratio* |
| May 20, 2002 | 1640 | 400,000 | — | — | — | — | — | — |
| | 1730 | — | 460 | — | — | — | — | — |
| | 1800 | — | 205 | 0.30 | 683 | — | — | — |
| | 2005 | 400,000 | — | — | — | — | — | — |
| May 21, 2002 | 0700 | 400,000 | 480 | 0.60 | 800 | — | — | — |
| | 0810 | — | 480 | 0.61 | 787 | — | — | — |
| | 1000 | 420,000 | — | — | — | — | — | — |
| | 1100 | — | 460 | 0.63 | 730 | 80 | 0.11 | 727 |
| | 1305 | 400,000 | 385 | 0.57 | 675 | — | — | — |
| | | | | | “baseline” | | | |
| | 1400 | 420,000 | — | — | — | — | — | — |
| | 1630 | 400,000 | 385 | 0.57 | 675 | 187 | 0.15 | 1,247 |
| | | | | | “baseline” | | | |
| | 1730 | — | — | — | — | 89 | 0.14 | 636 |
| | 2015 | — | — | — | — | 161 | 0.23 | 700 |
| | 2030 | 400,000 | — | — | — | — | — | — |
| | 2040 | — | 421 | 0.53 | 794 | — | — | — |
| | | | | | “baseline” | | | |
| May 22, 2002 | 0700 | 400,000 | — | — | — | — | — | — |
| | 0725 | — | — | 0.64 | — | — | — | — |
| | | | — | 0.60 | — | — | — | — |
| | 0753 | — | — | 0.54 | — | — | — | — |
| | 0758 | — | — | 0.58 | — | — | — | — |
| | 1045 | 420,000 | — | — | — | — | — | — |
| | 1656 | — | — | — | — | 66 | 0.09 | 733 |
| | 1831 | 420,000 | — | — | — | — | — | — |
| | 1845 | — | — | — | — | 73 | 0.10 | 730 |
| | | | | | | | | “expected ratio” |
| Mean | | 407,273 | 410 | 0.58 | 735 | 109 | 0.14 | 796 |
| SD | | 10,090 | 91 | 0.09 | 58 | 51 | 0.05 | 224 |
| n | | 11 | 8 | 11 | 7 | 8 | 6 | 6 |
| May 23, 2002 | 0700 | 400,000 | — | — | | — | — | — |
| | 0827 | 380,000 | — | — | | — | — | — |
| | 0832 | 375,000 | — | — | | — | — | — |
| | 1000 | 370,000 | @ 10% excess air | | | Rebum injector in Sect. 5 @ 6.0 lbs/hr | | |
| | | | — | — | | — | — | — |
| | 1145 | — | — | — | | 17 | 0.03 | 567 |
| | 1330 | — | — | — | | 205 | 0.30 | 683 |
| | | | | | | | | Ave: 625 |

*Ratio of: NOx Concentraion, ppm
Specific Mass Emissions, lbs NOx/MM BTU

TABLE 15
Proximate and Ultimate Analysis of 5 Selected Feedlot and Dairy Biomass
Samples (Hazen Research Inc.)

| | Panda Sample # | | | | | | |
|---|---|---|---|---|---|---|---|
| | A0035 | A0035 | A0044 | A0044 | A0201 | A0201 | A0213 |
| | TAES Sample # | | | | | | |
| | 170 | 170 | 171 | 171 | 172 | 172 | 173 |
| Parameter | As-Rec'd % | Dry, % | As-Rec'd % | Dry, % | As-Rec'd % | Dry, % | As-Rec'd % |
| Proximate (%): | | | | | | | |
| Moisture | 3.46 | 0.00 | 6.78 | 0.00 | 7.95 | 0.00 | 7.77 |
| Ash | 75.73 | 79.48 | 39.91 | 42.81 | 32.99 | 35.81 | 34.38 |
| Volatile | 18.44 | 18.10 | 46.65 | 50.04 | 49.38 | 53.62 | 40.05 |
| Fixed C | 1.37 | 1.42 | 0.66 | 7.15 | 9.73 | 10.57 | 7.80 |
| Total | 100 | 100 | 100 | 100 | 100 | 100 | 100 |
| Sulfur | 0.27 | 0.28 | 0.44 | 0.47 | 0.45 | 0.50 | 0.45 |
| HHV. BTU/lb | 1223 | 1266 | 4907 | 5263 | 5218 | 5657 | 5492 |
| MMF, BTU/lb | 7110 | 8841 | 8622 | 9794 | 9095 | 9240 | 8736 |
| MAF/DAF, BTU/lb | | 8172 | | 9204 | | 9827 | |
| Ultimate (%): | | | | | | | |
| Moisture | 3.46 | 0.00 | 5.78 | 0.00 | 7.95 | 0.00 | 7.77 |
| Carbon | 11.46 | 11.87 | 28.79 | 31.96 | 32.01 | 34.77 | 32.67 |
| Hydrogen | 0.94 | 0.98 | 5.57 | 3.85 | 3.88 | 4.00 | 3.67 |
| Nitrogen | 0.94 | 0.97 | 2.09 | 2.24 | 2.03 | 2.21 | 2.29 |
| Sulfur | 0.27 | 0.28 | 0.44 | 0.47 | 0.46 | 0.50 | 0.45 |
| Ash | 75.72 | 78,46 | 36.91 | 42.91 | 32.98 | 35.81 | 34.38 |
| Oxygen (diff.) | 6.20 | 6.42 | 17.42 | 18.89 | 20.91 | 22.71 | 18.57 |
| Total | 100.00 | 100.00 | 100.00 | 100.00 | 100.00 | 100.00 | 100.00 |
| Chlorine, Cl | 0.252 | 0.271 | 1.09 | 1.159 | 1.54 | 1.573 | 1.21 |
| Phosphorus | | 1.18 | | 4.47 | | 5.4 | |
| (Ash Basis), P205, % | | | | | | | |
| Phosphorus | | 0.94 | | 1.91 | | 1.93 | |
| (Dry Basis), P205, % | | | | | | | |
| Contaminants, | | | | | | | |
| Energy basis: | | | | | | | |
| Alka [symbol] /MM BTU | | | | | | | |
| Ash, lbs/MM BTU | 627.6 | | | 81.34 | | 63.19 | |
| SO2, lbs/MM BTU | 4.42 | | | 1.78 | | 1.76 | |
| DSCF/MM BTU | 15049 | | | 10416 | | 10218 | |

| | Panda Sample # | | | | | | |
|---|---|---|---|---|---|---|---|
| | A0213 | A0229 | A0229 | A0035-A0228 | | A0035-A0226 | |
| | TAES Sample # | | | | | | |
| | | | | 170-174 | | 170-174 | |
| | 173 | 174 | 174 | As-Rec'd, % | | Dry, % | |
| Parameter | Dry, % | As-Rec'd % | Dry, % | mean | [symbol] | mean | [symbol] |
| Proximate (%): | | | | | | | |
| Moisture | 0.00 | 4.34 | 0.00 | 8.08 | 0.00 | 0.00 | 0.00 |
| Ash | 37.28 | 73.55 | 76.90 | 51.51 | 21.84 | 54.46 | 21.84 |
| Volatile | 54.77 | 18.71 | 19.56 | 36.82 | 18.30 | 39.30 | 18.30 |
| Fixed C | 8.65 | 3.30 | 3.54 | 5.81 | 3.72 | 6.25 | 3.72 |
| Total | 100 | 100 | 100 | 100 | | 100 | |
| Sulfur | 0.40 | 0.18 | 0.17 | 0.38 | 0.15 | 0.38 | 0.15 |
| HHV. BTU/lb | 5955 | 1409 | 1966 | 3957 | 2323 | 3943 | 2323 |
| MMF, BTU/lb | 9872 | 7281 | 0240 | 7870 | 429 | 9437 | 429 |
| MAF/DAF, BTU/lb | 9404 | | 6778 | | | 9095 | 1513 |
| Ultimate (%): | | | | | | | |
| Moisture | 0.00 | 4.34 | 0.00 | 8.08 | 0.00 | 0.00 | 0.00 |
| Carbon | 35.42 | 11.46 | 11.98 | 23.48 | 12.19 | 25.20 | 12.19 |
| Hydrogen | 4.20 | 0.07 | 1.02 | 2.61 | 1.65 | 2.81 | 1.85 |
| Nitrogen | 2.46 | 1.17 | 1.22 | 1.70 | 0.68 | 1.62 | 0.88 |
| Sulfur | 0.49 | 0.18 | 0.17 | 0.38 | 0.15 | 0.35 | 0.15 |
| Ash | 37.28 | 73.58 | 78.60 | 51.51 | 21.64 | 54.46 | 21.84 |
| Oxygen (diff.) | 20.13 | 8.34 | 8.71 | 14.28 | 7.28 | 15.35 | 7.25 |
| Total | 100.00 | 100.00 | 100.00 | 100.00 | | 100.00 | |
| Chlorine, Cl | 1.312 | 0.303 | 0.317 | 0.00 | 0.03 | 0.85 | 0.83 |
| Phosphorus | 1.14 | | 1.21 | | | 2.08 | 2.08 |
| (Ash Basis), P205, % | | | | | | | |
| Phosphorus | 0.42 | | 0. [symbol] | | | 1.23 | 0.67 |
| (Dry Basis), P205, % | | | | | | | |
| Contaminants, | | | | | | | |
| Energy basis: | | | | | | | |
| Alka [symbol] /MM BTU | | | | | | | |
| Ash, lbs/MM BTU | 62.6 | | 490.99 | | | 174.53 | 211.15 |
| SO2, lbs/MM BTU | 1.64 | | 2.14 | | | 1.83 | 0.21 |
| DSCF/MM BTU | 10218 | | 11000 | | | 10633 | 704.61 |

[symbol]

indicates data missing or illegible when filed

TABLE 16
Elemental and Metals Analysis of Sample Ash from Feedlot
and Dairy Biomass Mixture (Hazen Research Inc.)
Ash Elemental Analysis (%), equal-weight-composite: Sample 172 & 174

| | Dry % |
|---|---|
| (Ash was calcined @ 1100 deg. F. | |
| (600 deg. C.) prior to analysis) | |
| Silicon, SiO2 | 89.36 |
| Aluminum, Al2O3 | 6.91 |
| Titanium, TiO2 | 0.31 |
| Iron, Fe2O3 | 2.5 |
| Calcium, CaO | 7.76 |
| Magnesium, MgO | 2.27 |
| Sodium, Na2O | 1.79 |
| Potassium, K2O | 5.81 |
| Phosphorus, P2O5 | 2.59 |
| Sulfur, SO3 | 1.68 |
| Chlorine, Cl | 1.55 |
| Carbon dioxide, CO2 | 0.07 |
| Total ash analysis | 102.6 |
| Metals in Ash, equal-weight-composite, mg/kg | |
| Arsenic | 12.10 |
| Barium | 412.00 |
| Cadmium | 3.00 |
| Chromium | 30.00 |
| Lead | <20 |
| Mercury | 0.01 |
| Selenium | <2 |
| Silver | <3 |
| Total metals in ash | 457.11 |

TABLE 17
Comparison of Dry Matter (DM) and Organic Matter (OM)
for Feedlot and Dairy Biomass using 3 Laboratory Approaches

| TAES/Arnar. | Hazen | GAN Lab | NIRS | Hazen | GAN Lab | NIRS |
|---|---|---|---|---|---|---|
| Sample # | DM | DM | DM | OM | OM | OM |
| 170 | 96.54 | 97.13 | 97.05 | 20.52 | 25.35 | 28.66 |
| 171 | 93.22 | ND | 95.14 | 57.19 | ND | 54.09 |
| 172 | 92.05 | 95.19 | 93.61 | 54.19 | 66.49 | 63.54 |
| 173 | 92.23 | 92.74 | 93.16 | 62.72 | 63.09 | 59.33 |
| 174 | 95.66 | 97.00 | 96.41 | 23.10 | 20.53 | 23.47 |

TABLE 18
Higher Heating Value (HHV BTY/lb) Measured vs. Predicted Using
Moisture (or dry matter), Ash (or dry matter/organic matter) Analyses

| TAES/ | | Predicted HHV from | | |
|---|---|---|---|---|
| Amar. | Hazen Analysis | Sweeten/Auvermann model | | |
| Sample # | HHV. as received | GAN Lab | Hazen | NIRS |
| 170 | 1223 | 2093 | 1684 | 2364 |
| 171 | 4907 | ND | 4532 | 4374 |
| 172 | 5216 | 5380 | 5022 | 5056 |
| 173 | 5492 | 4974 | 4917 | 4698 |
| 174 | 1498 | 1692 | 1878 | 1923 |

TABLE 19
Ash Content in HA-FB Before and After Multiple Passes Through
a DDS Technologies USA, Inc. Micrometric Separator

| Number of Passes of HA-FB | |
|---|---|
| Through Micrometric Separator | Mean % Ash d.b. |
| 0 | 54.55 |
| 1 | 41.56 |
| 2 | 29.71 |
| 3 | 26.24 |

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference is not an admission that it is prior art to the present invention, especially any reference that may have a publication date after the priority date of this application. The disclosures of all patents, patent applications, and publications cited herein are hereby incorporated by reference, to the extent they provide exemplary, procedural or other details supplementary to those set forth herein.

1. A method for the production of animal feedlot biomass for use as fuel in a reactor, the method comprising:
surfacing a feedlot with a feedlot surfacing material;
collecting an animal feedlot biomass from the feedlot; and
preparing the collected biomass for use as fuel.

2. The method of claim 1 wherein surfacing the feedlot comprises placing a surfacing material on the feedlot and compacting the surfacing material.

3. The method of claim 2 wherein the feedlot surfacing material is selected from the group consisting of fly ash, bottom ash from power plants, cements, and combinations thereof.

4. The method of claim 2 wherein placing the surfacing material on the feedlot comprises placing multiple layers of the surfacing material on the feedlot until a predetermined depth is achieved.

5. The method of claim 4 wherein the predetermined depth is between 3 and 8 inches.

6. The method of claim 1 wherein collecting the feedlot biomass comprises minimizing ash entrainment, water content, or both.

7. The method of claim 6 wherein minimizing minimizes ash entrainment, water content, or both comprises collecting the feedlot biomass during or after a relatively dry season.

8. The method of claim 6 wherein minimizing ash entrainment, water content, or both comprises collecting the feedlot biomass every one to two months during animal use of the feedlot.

9. The method of claim 6 wherein minimizing ash entrainment, water content, or both comprises collecting the uppermost ½ to ⅔ of the feedlot biomass.

10. The method of claim 6 wherein minimizing ash entrainment, water content, or both comprises attaining a biomass with less than 40% moisture by a method selected from the group consisting of providing good feedlot drainage, limiting absorption of precipitation, expediting natural evaporative drying, and combinations thereof.

11. The method of claim 1 further comprising partially composting the biomass in a compost pile.

12. The method of claim 11 wherein the compost pile further comprises mortality biomass, crop residues, and combinations thereof.

13. The method of claim 11 wherein partially composting the biomass comprises composting the biomass for about 20 days to about 60 days.

14. The method of claim 11 further comprising retarding the composting in a manner selected from the group consisting of allowing the partially composted biomass to attain a moisture content of less than 35%, collecting and bulk storing the partially composted biomass before it has finished composting, and combinations thereof.

15. The method of claim 11 wherein preparing the collected biomass for use as a fuel comprises air drying the partially composted biomass.

16. The method of claim 27 wherein air drying the partially composted biomass comprises placing the partially composted biomass in thin beds under dry storage.

17. The method of claim 15 wherein the partially composted biomass is air dried to a moisture content of equal to or less than about 10%.

18. The method of claim 15 wherein preparing the collected biomass for use as a fuel comprises pre-grinding the air dried biomass.

19. The method of claim 18 wherein air dried biomass is pre grinded to a median particle size characterized by from about 42% to about 51% passing through a 100 mesh.

20. The method of claim 18 wherein preparing the collected biomass for use as a fuel further comprises grinding the pre-ground biomass.

21. The method of claim 20 wherein grinding the pre-ground biomass comprises pulverizing the pre-ground biomass to a median particle size of about 50% passing through a 70 μm sieve.

22. The method of claim 1 wherein preparing the collected biomass for use as a fuel further comprises blending the biomass with pulverized coal.

23. The method of claim 22 wherein the pulverized coal is pulverized such that about 80% passes through a No. 200 standard mesh sieve and about 70% passes through a No. 325 mesh sieve.

24. The method of claim 22 wherein the pulverized coal to biomass ratio is from about 80:20 weight % to about 90:10 weight %.

25. The method of claim 22 wherein the reactor is a combustion or gasification reactor.

26. (canceled)

27. The method of claim 1 wherein preparing the collected biomass for use as fuel comprises reducing the amount of ash in the biomass.

28. The method of claim 27 wherein reducing the amount of ash comprises separating ash from the biomass with a separator.

29. The method of claim 27 wherein the separator is a micrometric separator.

30. The method of claim 28 wherein separating ash from the biomass with a separator comprises passing the biomass through the separator a plurality of times.

31. The method of claim 28 wherein the separator comprises a plurality of separators arranged in series.

32. The method of claim 1 further comprising field testing the collected biomass for combustion quality.

33. The method of claim 32 wherein field testing the biomass comprises a test selected from the group consisting of bulk density tests, colorimetric analyses, particle size analyses, protein content analyses, and combinations thereof.

34. The method of claim 32 wherein field testing the collected biomass comprises using near-infrared spectroscopy to determine the ash content and the moisture content of the feedlot biomass.

35. The method of claim 1 wherein the reactor comprises a thermal conversion reactor or a bioconversion reactor.

36. The method of claim 35 wherein the animal feedlot biomass undergoes combustion, gasification, pyrolysis, or a combination thereof within the thermal conversion reactor.

37. The method of claim 35 wherein the animal feedlot biomass undergoes anaerobic digestion, fermentation, or both within the bioconversion reactor.

38. The method of claim 1 further comprising using the biomass as co-firing fuel in a reactor of a coal combustion system, wherein fly ash, bottom ash, or both formed in the reactor upon combustion are used as feedlot surfacing material.

39. The method of claim 1 further comprising adjusting a feed ration to alter the amount of at least one mineral in the feedlot biomass.

40. The method of claim 39 wherein the amount of the at least one mineral is altered to enhance reactor performance.

41. The method of claim 39 wherein adjusting the feed ration comprises decreasing the phosphorus content of the feed ration.

42. The method of claim 39 wherein adjusting the feed ration comprises decreasing the content in the feed ration of a component selected from the group consisting of crude proteins, salts, and combinations thereof.

43. The method of claim 1 wherein the biomass has a higher heating value of greater than about 8000 BTU/lb on a dry ash free (DAF) basis.

44. The method of claim 43 wherein the ash content of the collected biomass is less than the ash content of conventionally collected biomass.

45. The method of claim 44 wherein the ash content is less than about 30% on a wet basis.

46. The method of claim 44 wherein the ash content is less than about 30% on a dry basis.

47. The method of claim 1 wherein at least one parameter of the collected biomass is increased relative to conventionally collected biomass, the parameter selected from the group consisting of higher heating value, volatile matter, fixed carbon and combinations thereof.

48. The method of claim 47 wherein the higher heating value of the biomass is increased by about 50% or more.

49. The method of claim 47 wherein the volatile solids of the biomass is increased by at least 40%.

50. The method of claim 47 wherein the fixed carbon of the biomass is increased by at least 50%.

51. The method of claim 47 wherein the total carbon of the biomass is increased by at least 50%.

52. A method for the reduction of NO emissions from a coal-fired power plant comprising:
preparing the fuel according to claim 1 and reducing the NO emissions of the coal fired power plant by using the fuel as a co firing fuel the coal fired power plant.

53. A method for the reduction of the emission of at least one heavy metal from a carbonaceous feed combustion process, the method comprising:
preparing the fuel according to claim 1 and reducing the emission of the at least one heavy metal by co-firing the fuel with carbonaceous feed.

54. The method of claim 53 wherein the at least one heavy metal is mercury.
